(19) United States
(12) Patent Application Publication
(10) Pub. No.: US 2013/0303788 A1
(43) Pub. Date: Nov. 14, 2013

(54) METHOD OF PRODUCING BIPHENOLIC COMPOUND, NOVEL BIPHENYL COMPOUND AND SYNTHESIS METHOD THEREOF, AND PHARMACEUTICAL COMPOSITION FOR TREATING PARKINSON'S DISEASE

(71) Applicants: Ming-Huan Chan, Yongjing Township (TW); Chinpiao Chen, Soufeng (TW); Hwei-Hsien Chen, Zhunan (TW)

(72) Inventors: Ming-Huan Chan, Yongjing Township (TW); Chinpiao Chen, Soufeng (TW); Hwei-Hsien Chen, Zhunan (TW)

(73) Assignee: Tzu Chi University, Hualien City (TW)

(21) Appl. No.: 13/616,042

(22) Filed: Sep. 14, 2012

(30) Foreign Application Priority Data
May 14, 2012 (TW) 101117093

(51) Int. Cl. C07C 37/11; C07C 39/15; C07C 39/21; C07D 309/12

(52) U.S. Cl. 549/416; 568/730

(57) ABSTRACT

A method of producing honokiol and analogues thereof, and novel intermediates prepared by virtue thereof are disclosed herein. A pharmaceutical composition for treating Parkinson's disease, which contains honokiol and/or the analogues thereof, is also disclosed herein.

### CROSS-REFERENCE TO RELATED APPLICATION

This application claims priority of Taiwanese application no. 101117093, filed on May 14, 2012.

### BACKGROUND OF THE INVENTION

1. Field of the Invention

This invention relates to a novel method of producing honokiol and analogues thereof, and new intermediate compounds prepared using the aforesaid method. This invention also relates to a pharmaceutical composition for treating Parkinson's disease, which comprises honokiol and/or the analogues thereof.

2. Description of the Related Art

Numerous studies have indicated that polyphenols have in vitro and in vivo activities against oxidative stress associated with certain human and animal diseases (Singh M. et al. (2008), J. Agrc. Food Chem., 56:4855-4873). Furthermore, polyphenols are proven to be able to protect neuronal cells in various in vivo and in vitro models by acting on different intracellular targets (Ramassamy C. (2006), Eur. J. Pharm., 545:51-64). Biphenolic neolignans are such polyphenols.

Honokiol and magnolol (the biphenolic neolignans shown below) are isomers and are bioactive components isolated from the bark of Magnolia officinalis (Fujita M. et al. (1973), Yakugaku Zasshi, 93: 429-434; Li A. J. (1985), Zhong Yao Tong Bao, 10: 10-13).

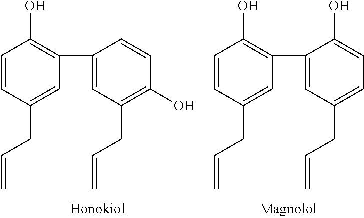

Honokiol and magnolol are expected to be potential therapeutic agents for neurodegenerative diseases since honokiol and magnolol are able to exert neuroprotective effects via their anti-oxidative abilities and antagonist actions against excitotoxicity induced by excitatory amino acids (Lin Yi-Ruu et al. (2006), Eur. J. Pharm., 537: 64-69). In addition, honokiol and magnolol have been proven to be effective in alleviating inflammatory pain in an animal model (Lin Yi-Ruu et al. (2007), Life Sciences, 81:1071-1078).

Parkinson's disease (PD) is a neurodegenerative disorder characterized by loss of the pigmented dopaminergic (DA) neurons in the substantia nigra pars compacta (SNpc), which in turn leads to loss of striatal dopamine. Scientists have reported that: PD might be attributed to oxidative stress, inflammation, mitochondrial dysfunction, excitotoxicity, etc., and is hence a multifactorial disorder (Armstrong R. J. et al. (2001), Lancet, 358:1174-1176; Jenner P. et al. (2006), Neurology, 66:S24-36). Since honokiol and magnolol have neuroprotective and anti-inflammatory effects, the same are anticipated to be useful for treating PD. In WO 2008074896 A1, it is even presumed that honokiol and magnolol may be used to treat PD since the same can reduce or abolish addiction of methamphetamine, which leads to depletion of dopamine in the non-motor portion of the basal ganglia system. However, the prior art lacks direct evidence showing that honokiol and magnolol are effective in treating PD.

Accordingly, comprehensive research is still required to prove that honokiol and magnolol are effective in treating PD since PD is multifactorial. In addition to in vitro models for oxidative stress and neurotoxicity (resulting in neuronal death), the applicant has employed an animal model of PD, which possesses behavioral and pathological symptoms similar to those of patients with PD, so as to show that honokiol has a therapeutic effect on PD. The applicant has further found that an analogue of honokiol also exhibits a therapeutic effect on PD, thereby indicating that honokiol and analogues thereof can be used to treat PD.

Unfortunately, the conventional method for extracting biphenolic neolignans from the bark of Magnolia species is still unsatisfactory. Thus, attempts to synthesize biphenolic neolignans have been made. The conventional methods for synthesizing honokiol and analogues thereof normally employ Suzuki reaction (i.e., a chemical reaction between an aryl- or vinyl-boronic acid or ester and an aryl- or vinyl halide, which is catalyzed via a palladium complex). For example, 2-bromo-4-allyl anisole (shown below) and 4-(tetrahydro-2H-pyran-2-yloxy)-phenylboronic acid pinacol ester (not shown) may be subjected to Suzuki reaction to form a biphenyl compound. However, two inseparable biphenyl isomers (shown below) are formed instead due to the isomerization of the allyl substituent.

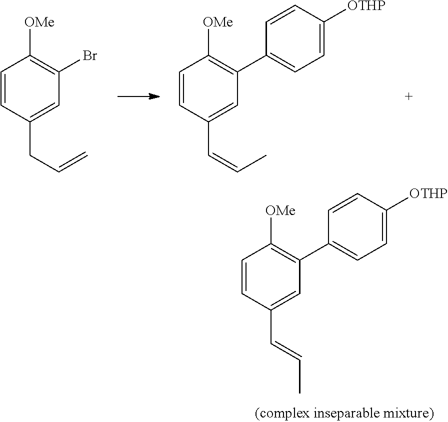

Consequently, a desired honokiol-type compound can only be synthesized in unsatisfactory yield using the aforesaid method. Moreover, a boronic acid having an allylanisole portion (shown below) and a bromophenol compound (shown below) may be subjected to Suzuki reaction to form a biphenyl compound. Similarly, two biphenyl isomers (see below) are formed instead due to the isomerization of the allyl substituent, thereby reducing the overall yield of a desired honokiol-type compound.

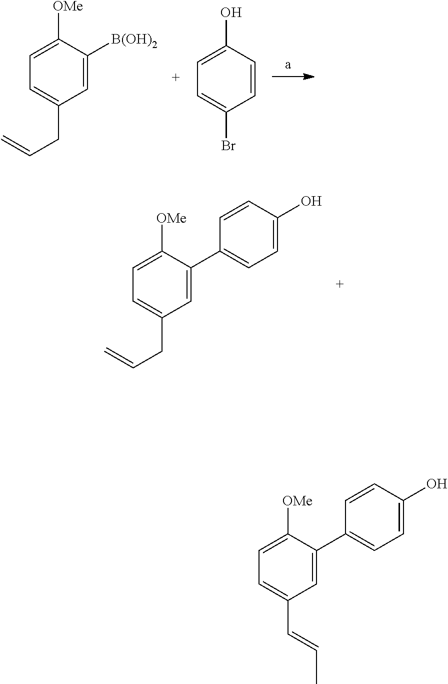 [SMILES: C=CCc1ccc(OC)c(B(O)O)c1 ; Oc1ccc(Br)cc1 ; C=CCc1ccc(OC)c(-c2ccc(O)cc2)c1 ; C/C=C/c1ccc(OC)c(-c2ccc(O)cc2)c1]

One might contemplate that an allyl substituent may be dispensed with so as to prevent isomerization, like Esumi T. et al. In Esumi et al. (2004), Bioorg. & Med. Chem. Lett, 14:2621-2625, an arylboronic pinacol ester without an allyl substituent (shown below) and an aryl bromide compound without an allyl substituent (shown below) were subjected to Suzuki reaction so as to prepare a biphenyl compound (87% yield), which was later used to form honokiol, but the overall yield of honokiol was only 21% since the arylboronic pinacol ester and the aryl bromide compound must be respectively prepared from 5-bromosalicylic acid (shown below) and 4-hydroxybenzoic acid (shown below) using arduous steps.

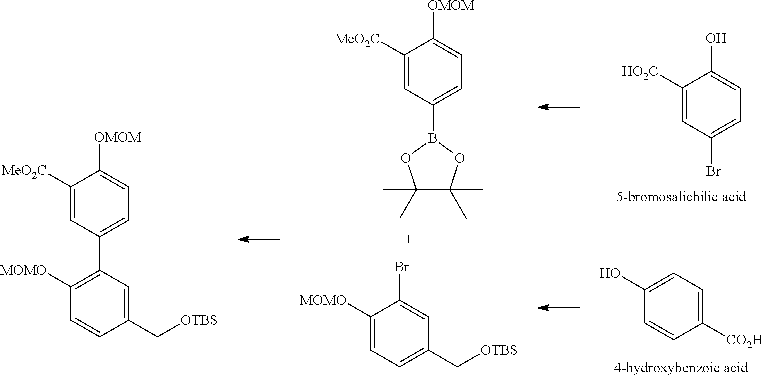

Thus, the applicant has conceived of a novel method for synthesizing honokiol and analogues thereof, which comprises subjecting an anisole compound without an allyl substituent and an arylboronic compound without an allyl substituent to Suzuki reaction so as to prevent isomerization. The anisole compound without an allyl substituent and the arylboronic compound without an allyl substituent selected by the applicant can be simply prepared with good yield.

### SUMMARY OF THE INVENTION

Therefore, according to a first aspect, this invention provides a method of producing a biphenolic compound of formula (I):

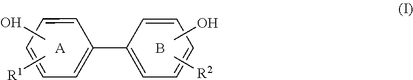

wherein R¹ in ring A and R² in ring B independently represent a C₁-C₁₂ alkyl group, a C₂-C₁₂ alkenyl group, or a C₂-C₁₂ alkynyl group;

the method comprising:

subjecting an anisole compound of formula (II) and an arylboronic compound of formula (III) to Suzuki reaction so that a biphenyl compound of formula (I′) is formed:

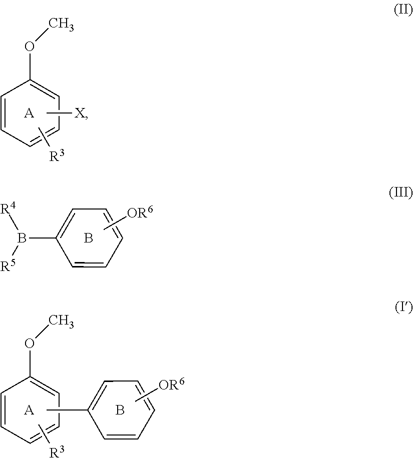

wherein, in formula (II), X represents halogen, and, in formulas (II) and (I′), R³ is H, an optionally substituted C₁-C₁₂ alkyl group, a C₂ or C₄-C₁₂ terminal alkenyl group, or a —(CH₂)ₙ—CH(OH)CH₂OH group, n being an integer from 1-10;
wherein, in formula (III), R⁴ and R⁵ represent OH, or R⁴ and R⁵ together with the boron atom to which R⁴ and R⁵ are attached form boronic ester; and, in formulas (III) and (I′), R⁶ represents tetrahydropyranyl,
removing R⁶ from the biphenyl compound of formula (I′), followed by attaching a R⁷ group to the ring B, R⁷ having the same definition as R²; and
converting the methoxy group in ring A to a hydroxy group.

In a second aspect, this invention provides the aforesaid biphenyl compound of formula (I′).

In a third aspect, this invention provides a method of producing the aforesaid biphenyl compound of formula (I′), which comprises subjecting the aforesaid anisole compound of formula (II) and the aforesaid arylboronic compound of formula (III) to Suzuki reaction.

In a fourth aspect, this invention provides a pharmaceutical composition for treating Parkinson's disease, which comprises the aforesaid biphenolic compound of formula (I).

### BRIEF DESCRIPTION OF THE DRAWINGS

The above and other objects, features and advantages of this invention will become apparent with reference to the following detailed description and the preferred embodiments taken in conjunction with the accompanying drawings, in which:

FIG. 1 shows a synthesis scheme for 5,3′-diallyl-biphenyl-2,4′-diol (honokiol), i.e., product 1 or compound 1, in which Me stands for methyl, and THP stands for tetrahydropyranyl;

FIG. 2 shows a synthesis scheme for 3′-allyl-5-propyl-biphenyl-2,4′-diol, i.e., product 2 or compound 13, and for 5,3′-dipropyl-biphenyl-2,4′-diol, i.e., product 3 or compound 14, in which Me stands for methyl, and THP stands for tetrahydropyranyl;

FIG. 3 shows a synthesis scheme for 5-allyl-3′-propyl-biphenyl-2,4′-diol, i.e., product 4 or compound 19, in which Me stands for methyl, and Ms stands for methanesulfonyl;

FIG. 4 shows a synthesis scheme for 3′-allyl-5-prop-2-ynyl-biphenyl-2,4′-diol, i.e., product 5 or compound 25, in which Me stands for methyl, and MOM stands for methoxymethyl;

FIG. 5 shows a synthesis scheme for 5-propyl-3′-prop-2-ynyl-biphenyl-2,4′-diol, i.e., product 6 or compound 29, in which Me stands for methyl, and MOM stands for methoxymethyl;

FIG. 6 shows a synthesis scheme for 2,3′-diallyl-biphenyl-3,4′-diol, i.e., product 7 or compound 34, and for 4,3′-diallyl-biphenyl-3,4′-diol, i.e., product 8 or compound 35, in which Me stands for methyl, and THP stands for tetrahydropyranyl;

FIG. 7 shows a synthesis scheme for 3′-allyl-2-propyl-biphenyl-4,4′-diol, i.e., product 9 or compound 42, in which Me stands for methyl, THP stands for tetrahydropyranyl, and Ms stands for methanesulfonyl;

FIG. 8 shows a synthesis scheme for 2,3′-dipropyl-biphenyl-4,4′-diol, i.e., product 10 or compound 53, in which Me stands for methyl;

FIG. 9 shows the effects of products 1, 2, 3, 4, 5, 7, and 3,3′-diallyl-biphenyl-4,4′-diol (compound 55) on oxidative stress (n=3 for the respective biphenolic compound of formula (I), the normal control, and the pathological control), in which “*” indicates p<0.05 (ANOVA) when the pathological control is compared with the normal control, “#” indicates p<0.05 (ANOVA) when the SH-SY5Y human neuroblastoma cells treated with the respective biphenolic compound of formula (I) are compared with the pathological control, HP stands for hydroperoxide, pathol stands for pathological, prod stands for product, and comp stands for compound;

FIG. 10 shows the effects of products 1, 2, 3, 4, 5, 7, and 3,3′-diallyl-biphenyl-4,4′-diol (compound 55) on neurotoxicity (n=4 for each concentration of the respective biphenolic compound of formula (I), the normal control, and the pathological control), in which “*” indicates p<0.05 (ANOVA) when the pathological control is compared with the normal control, “#” indicates p<0.05 (ANOVA) when the SH-SY5Y human neuroblastoma cells treated with the respective biphenolic compound of formula (I) are compared with the pathological control, MPP+ stands for 1-methyl-4-phenylpyridinium, pathol stands for pathological, prod stands for product, and comp stands for compound;

FIG. 11 shows the effect of 14 days of the subchronic treatment with product 1 on the rotational behavior induced by 6-OHDA (n=4-6 for the sham operated control, and the pathological control, 5 mg/kg of product 1, and 10 mg/kg of product 1), in which “**” indicates p<0.01 (ANOVA and the Student-Newman-Keuls post-hoc test) when the NMRI mice treated with product 1 are compared with the pathological control;

FIG. 12 are photomicrographs illustrating the effect of 14 days of the subchronic treatment with product 1 on reduction in TH expression of the striatum induced by 6-OHDA, in which “intact” indicates that 6-OHDA or the vehicle was not injected into the corresponding striatum, “lesion” indicates that 6-OHDA or the vehicle was injected into the corresponding striatum, and the scale bar represents 0.8 mm;

FIGS. 13(A) and 13(B) respectively show the effects of 14 days of the subchronic post-treatments with product 1 and product 2 on the rotational behavior induced by 6-OHDA (FIG. 13(A): n=4 for the pathological control, n=7 for 1 mg/kg of product 1, n=8 for O_{—}5 mg/kg of product 1, and n=4 for 0.1 mg/kg of product 1; FIG. 13(B): n=12 for the pathological control, n=13 for 0.05 mg/kg of product 2, n=14 for 0.1 mg/kg of product 2, n=16 for 0.5 mg/kg of product 2, and n=6 for 1 mg/kg of product 2), in which “*” indicates p<0.05 (ANOVA and the Student-Newman-Keuls post-hoc test) when the NMRI mice treated with product 1 or product 2 are compared with the pathological control;

FIG. 14 are photomicrographs illustrating the effect of 14 days of the subchronic post-treatment with product 1 on TH-ir fiber loss in the striatum and the substantia nigra (SN) induced by 6-OHDA, in which “intact” indicates that 6-OHDA or the vehicle was not injected into the corresponding striatum, “lesion” indicates that 6-OHDA or the vehicle was injected into the corresponding striatum, the scale bar represents 0.8 mm, and SN stands for substantia nigra;

FIGS. 15(A) and 15(B) respectively show the effect of 14 days of the subchronic post-treatment with product 1 on TH-ir fiber loss in the striatum and the substantia nigra induced by 6-OHDA (n=4 for the sham operated control, the pathological control, 0.5 mg/kg of product 1, 1 mg/kg of product 1, and 5 mg/kg of product 1), in which “*” indicates p<0.05 (ANOVA and the Student-Newman-Keuls post-hoc test) when the pathological control is compared with the sham operated control, “#” indicates p<0.05 (ANOVA and the Student-Newman-Keuls post-hoc test) when the NMRI mice treated with product 1 are compared with the pathological control, and SN stands for substantia nigra;

FIG. 16 are photomicrographs illustrating the effect of 14 days of the subchronic post-treatment with product 2 on TH-ir fiber loss in the striatum and the substantia nigra (SN) induced by 6-OHDA, in which “intact” indicates that 6-OHDA was not injected into the corresponding striatum, “lesion” indicates that 6-OHDA was injected into the corresponding striatum, the scale bar represents 0.8 mm, and SN stands for substantia nigra;

FIG. 17 shows the effect of 14 days of the subchronic post-treatment with product 1 on iNOS expression in the striatum induced by 6-OHDA (n=4 for the sham operated control, the pathological control, 0.5 mg/kg of product 1, 1 mg/kg of product 1, and 5 mg/kg of product 1), in which “***” indicates p<0.001 (ANOVA and the Student-Newman-Keuls post-hoc test) when the pathological control is compared with the sham operated control, and “###” indicates p<0.001 (ANOVA and the Student-Newman-Keuls post-hoc test) when the NMRI mice treated with product 1 are compared with the pathological control;

FIGS. 18(A) and 18(B) respectively show the effect of the pretreatment with product 1 on incoordination induced by 6-OHDA 1 day and 7 days after the 6-OHDA injection (n=8 for the sham operated control, the pathological control, and product 1), in which “***” indicates p<0.001 (ANOVA and the Student-Newman-Keuls post-hoc test) when the pathological control is compared with the sham operated control, and “###” indicates p<0.001 (ANOVA and the Student-Newman-Keuls post-hoc test) when the NMRI mice treated with product 1 are compared with the pathological control; and

FIGS. 19(A) and 19(B) respectively show the effects of the 7-day and 14-day post-treatments on incoordination induced by 6-OHDA (n=6 for the sham operated control, the pathological control, and product 1), in which “***” indicates p<0.001 (ANOVA and the Student-Newman-Keuls post-hoc test) when the pathological control is compared with the sham operated control, and “###” indicates p<0.001 (ANOVA and the Student-Newman-Keuls post-hoc test) when the NMRI mice treated with 21) product 1 are compared with the pathological control.

### DETAILED DESCRIPTION OF THE INVENTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless defined otherwise, all technical and scientific terms used herein have the meaning commonly understood by a person skilled in the art to which this invention belongs. One skilled in the art will recognize many methods and materials similar or equivalent to those described herein, which could be used in the practice of the present invention. Indeed, the present invention is in no way limited to the methods and materials described. For clarity, the following definitions are used herein.

The term “Suzuki reaction” as used herein refers to a chemical reaction between an aryl- or vinyl-boronic acid or ester and an aryl- or vinyl halide, which is catalyzed via a palladium complex.

The term “halogen” as used herein refers to fluorine, chlorine, bromine, or iodine.

The term “optionally substituted alkyl” as used herein alone or as part of another group refers to a straight or branched saturated monovalent hydrocarbon group.

The term “terminal alkenyl” as used herein alone or as part of another group refers to an alkenyl group having the double bond that is positioned between the penultimate and terminal carbons thereof.

In order to efficiently synthesize honokiol and analogues thereof, the applicant has conceived of a novel method for producing honokiol and analogues thereof. The applicant selected an anisole compound without an allyl substituent and an arylboronic compound without an allyl substituent as two starting materials for Suzuki reaction so as to prevent isomerization. The anisole compound without an allyl substituent and the arylboronic compound without an allyl substituent selected by the applicant can be simply prepared with good yield.

Accordingly, this invention provides a method of producing a biphenolic compound of formula (I):

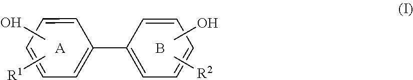

wherein R¹ in ring A and R² in ring B independently represent a C₁-C₁₂ alkyl group, a C₂-C₁₂ alkenyl group, or a C₂-C₁₂ alkynyl group;

the method comprising:

subjecting an anisole compound of formula (II) and an arylboronic compound of formula (III) to Suzuki reaction so that a biphenyl compound of formula (I′) is formed:

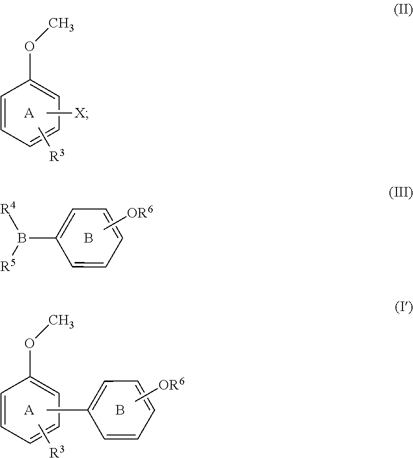

wherein, in formula (II), X represents halogen, and, in formulas (II) and (I′), R³ is H, an optionally substituted C₁-C₁₂ alkyl group, a C₂ or C₄-C₁₂ terminal alkenyl group, or a —(CH₂)ₙ—CH(OH)CH₂OH group, n being an integer from 1-10;
wherein, in formula (III), R⁴ and R⁵ represent OH, or R⁴ and R⁵ together with the boron atom to which R⁴ and R⁵ are attached form boronic ester; and, in formulas (III) and (I′), R⁶ represents tetrahydropyranyl,

removing R⁶ from the biphenyl compound of formula (I′), followed by attaching a R⁷ group to the ring B, R⁷ having the same definition as R²; and

converting the methoxy group in the ring A to a hydroxy group.

When R³ is H or the —(CH₂)ₙ—CH(OH)CH₂OH group, the method further comprises converting R³ to R¹.

Preferably, R¹ and R² may independently represent propyl, propenyl, propynyl, 2-methylpropyl, 2,2-dimethylpropyl, butyl, pentyl, hexyl, 3-butenyl, or 4-pentenyl.

Preferably, R³ is selected from the group consisting of propyl, 2,3-dihydroxypropyl, 2-methylpropyl, 2,2-dimethylpropyl, butyl, pentyl, hexyl, 3-butenyl, and 4-pentenyl. Preferably, X is Br. Examples of the anisole compound include 3-(3-bromo-4-methoxy-phenyl)-propane-1,2-diol, 2-bromo-4-propyl anisole, 3-bromo anisole, 4-bromo-5-propyl anisole, and 3-(2-bromo-5-methoxy-phenyl)-propane-1,2-diol.

Preferably, R⁴ and R⁵ together with the boron atom to which R⁴ and R⁵ are attached form boronic acid pinacol ester. In a preferred embodiment of this invention, the arylboronic compound is 4-(tetrahydro-2H-pyran-2-yloxy)-phenylboronic acid pinacol ester.

The applicant synthesized 10 biphenolic compounds of formula (I), including honokiol, via the method of the present invention, and found that the 10 biphenolic compounds of formula (I) were synthesized with good yield. Thus, the method of the present invention is suitable for large-scale industrial production.

Since the Suzuki coupling step of the method according to this invention (i.e., the Suzuki reaction employing the anisole compound without an allyl substituent and the arylboronic compound without an allyl substituent) is novel, this invention also provides the aforesaid biphenyl compound of formula (I′). The aforesaid biphenyl compound of formula (I′) is a stable intermediate.

Examples of the aforesaid biphenyl compound of formula (I′) include 3-[6-methoxy-4′-(tetrahydro-pyran-2-yloxy)-biphenyl-3-yl]-propane-1,2-diol, 2-(2′-methoxy-5′-propyl-biphenyl-4-yloxy)-tetrahydro-pyran, 2-(3′-methoxy-biphenyl-4-yloxy)-tetrahydro-pyran, 2-(4′-methoxy-2′-propyl-biphenyl-4-yloxy)-tetrahydro-pyran, and 2-[4-methoxy-4′-(tetrahydro-pyran-2-yloxy)-biphenyl-3-yl]-propane-1,2-diol.

In order to prove that honokiol and analogues thereof are effective in treating Parkinson's disease (PD), the applicant first selected 6 out of the previously synthesized 10 biphenolic compounds of formula (I), including honokiol, i.e., products 1, 2, 3, 4, 5, 7, and synthesized an additional biphenolic compound of formula (I) (i.e., compound 55) via a conventional method, so as to conduct two in vitro experiments, which were respectively designed to investigate the effects of the test biphenolic compounds on oxidative stress and neurotoxicity. The test biphenolic compounds were verified to be capable of protecting neuronal cells from being damaged by oxidative stress and neurotoxicity. Therefore, honokiol and analogues thereof can exert neuroprotective effects against oxidative stress and neurotoxicity, and are hence expected to be useful in treating PD.

The applicant further performed an in vivo experiment so as to confirm the therapeutic effects of honokiol and analogues thereof on PD. A mouse model of PD, which was prepared via injection of neurotoxin 6-hydroxydopamine (6-OHDA) into a unilateral striatum, was used. Two of the previously synthesized 10 biphenolic compounds of formula (I), including honokiol, i.e., product 1 and product 2, were used in the in vivo experiment. The two test biphenolic compounds of formula (I) prevented loss of dopaminergic (DA) neurons and reduced apomorphine-induced rotational behaviors. Honokiol and analogues thereof hence have neuroprotective effects on DA neurons and are able to ameliorate symptoms of PD.

Accordingly, this invention provides a pharmaceutical composition for treating Parkinson's disease, which comprises the aforesaid biphenolic compound of formula (I).

Examples of the aforesaid biphenolic compound of formula (I) include the group consisting of 5,3′-diallyl-biphenyl-2,4′-diol, 3′-allyl-5-propyl-biphenyl-2,4′-diol, 5,3′-dipropyl-biphenyl-2,4′-diol, 5-allyl-3′-propyl-biphenyl-2,4′-diol, 3′-allyl-5-prop-2-ynyl-biphenyl-2,4′-diol, 2,3′-diallyl-biphenyl-3,4′-diol, and 3,3′-diallyl-biphenyl-4,4′-diol. In a preferred embodiment of this invention, the aforesaid biphenolic compound of formula (I) is 5,3′-diallyl-biphenyl-2,4′-diol. In another preferred embodiment of this invention, the aforesaid biphenolic compound of formula (I) is 3′-allyl-5-propyl-biphenyl-2,4′-diol.

The pharmaceutical composition according to this invention can be formulated into a suitable dosage form for parenteral or oral administration, which includes, but is not limited to, injections (e.g., sterile aqueous solutions or dispersions), sterile powder, tablets, troches, pills, capsules, caplets, lozenges, pellets, dispersible powder or granules, solutions, suspensions, emulsions, syrup, elixir, slurry, and the like.

The parenteral route of administration suitable for the pharmaceutical composition according to this invention includes, but is not limited to, intraperitoneal injection, subcutaneous injection, intramuscular injection, intravenous injection, intraarterial injection, intrathecal injection, intracerebroventricular injection, and intracranial injection. In a preferred embodiment of this invention, the parenteral route of administration is intraperitoneal injection.

The pharmaceutical composition according to this invention can additionally comprise a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, gelling agents, preservatives, wetting agents, lubricants, diluents, absorption delaying agents, liposome, sweetening agents, flavoring agents, coloring agents, and the like.

If necessary, the pharmaceutical composition according to this invention may comprise one or more of the following pharmaceutically acceptable solvents: water, normal saline, phosphate buffered saline (PBS), sugar-containing solutions, aqueous solutions containing alcohol, oil, glycerol, organic solvents, and liposome.

The dosage and the frequency of administration of the biphenolic compounds of formula (I) may vary depending on the following factors: the severity of the disease to be treated, the route of administration, and the weight, age, physical condition and response of the subject to be treated. For instance, the daily dosage of the biphenolic compounds of formula (I) may be 10.8-21.6 mg per 60 kilograms of the body weight, and may be administered in a single dose or in several doses.

The present invention will be described in more detail with reference to the following examples, which are given for the purpose of illustration only and are not intended to limit the scope of the present invention.

### EXAMPLES

### Synthesis Examples

In order to prove that the biphenolic compounds of formula (I) can be synthesized with good yield using the method of the present invention, the following syntheses were conducted.

### General Procedures:

Silica gel column chromatography was conducted using MN silica gel 60 (70-230 mesh, Macherey-Nage).

Melting point was determined using a Yanagimoto Micro Melting Point Apparatus (Model-S3).

¹H-NMR and ¹³C-NMR spectra were recorded on a Bruker Avance II 400 spectrometer (400 MHz) and a Bruker Avance DPX 300 spectrometer (300 MHz), in which CDCl₃ was used as a solvent for the NMR measurements, and the chemical shift (δ) is expressed in ppm relative to a standard.

IR spectra were obtained using a Perkin-Elmer Spectrum One Spectrometer.

EIMS spectra and HRMS-EI spectra were recorded on a Finnigan/Thermo Quest MAT 95XL Mass Spectrometer.

### Synthesis Ex. 1

### 5,3′-Diallyl-biphenyl-2,4′-diol (Product 1 or Compound 1)

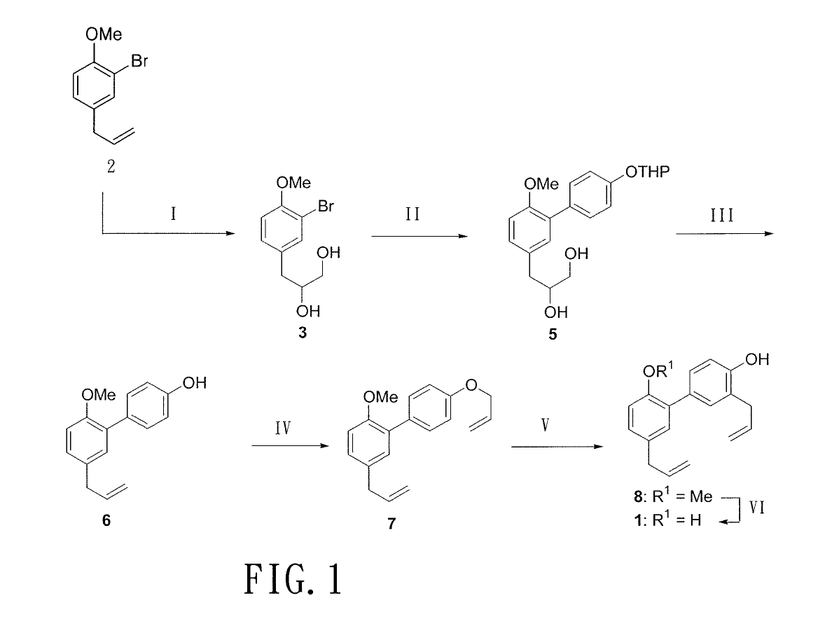

Synthesis of product 1 (i.e., honokiol) is outlined in FIG. 1.

### Step I. Preparation of 3-(3-bromo-4-methoxy-phenyl)-propane-1,2-diol (Compound 3)

4-allylanisole (Aldrich) was used to synthesize 2-bromo-4-allyl anisole (compound 2) in a two-step process (i.e., sequential hydrogenation and bromination).

738 mg of the resultant 2-bromo-4-allyl anisole (compound 2) was dissolved in 27 mL of an acetone-water (8:1) solution, followed by adding 421 mg of N-Methylmorpholine-N-oxide (NMO) and 0.11 mL of a 2.5% solution of OsO₄ in 2-methyl-2-propanol under argon atmosphere. The resultant mixture was stirred at room temperature for 24 h (TLC monitoring), followed by adding 10 mL of an aqueous Na₂SO₃ solution (10%). Stirring was conducted at room temperature for 30 minutes. An evaporation treatment was performed under vacuo. The resultant residue was subjected to an extraction treatment using 30 mL of ethyl acetate (EtOAc). The extract thus obtained was washed with 10 mL of brine thrice. The resultant organic layer was dried over MgSO₄, followed by a concentration process. Subsequently, the residue thus acquired was subjected to a purification treatment employing silica gel column chromatography (EtOAc-hexane (1:1) as a mobile phase). Thus, 3-(3-bromo-4-methoxy-phenyl)-propane-1,2-diol (compound 3) as a solid was formed (91% yield).

### Detected Properties of the Title Compound:

M.p.: 88-90° C. ¹H NMR (400 MHz, CDCl₃, δ): 7.41 (d, J=2.1 Hz, 1H), 7.12 (m, 1H), 6.83 (d, J=8.4 Hz, 1H), 3.87 (s, 3H), 3.66 (dd, J=3.2, 11.2 Hz, 1H), 3.47 (dd, J=7.0, 11.2 Hz, 1H), 2.67 (m, 2H), 2.35 (brs, 2H). ¹³C NMR (100.6 MHz, CDCl₃, δ): 154.7, 133.9, 131.4, 129.3, 112.0, 111.7, 72.9, 65.9, 56.3, 38.4. IR (KBr): 3379, 3076, 3016, 2950, 2840, 1736, 1604, 1511, 1492, 1462, 1440, 1253, 1229, 1042, 831 cm⁻¹. EIMS (70 eV) m/z: 262.2 (M+2, 31.0), 260.2 (M⁺, 31.5), 201.1 (100), 199.1 (99.2), 121.1 (65.1), 77.1 (50.0), 185.1 (36.4), 260.2 (31.56), 262.2 (31.00). HRMS-EI (m/z): M⁺ calc. for C₁₀H₁₃BrO₃: 260.0048. found: 260.0041.

### Step II. Preparation of 3-[6-methoxy-4′-(tetrahydro-pyran-2-yloxy)-biphenyl-3-yl]-propane-1,2-diol (Compound 5)

2.5 g of the 3-(3-Bromo-4-methoxy-phenyl)-propane-1,2-diol (compound 3) as obtained in Step I of this example and 3.5 g of 4-(tetrahydro-2H-pyran-2-yloxy)-phenylboronic acid pinacol ester (compound 4) were dissolved in 66 mL of 1,2-dimethoxyethane (DME). 350 mg of PdCl₂(dppf).CH₂Cl₂ as a catalyst (dppf: I,I′-Bis(diphenylphosphino)ferrocene) was added to the resultant solution under argon, followed by adding 30 mL of an aqueous Na₂CO₃ solution (2 M). The thus obtained mixture was heated with stirring at 80° C. for 18 hours (TLC monitoring). Afterwards, an evaporation process in vacuo was conducted. The resultant residue was subjected to an extraction treatment using 100 mL of EtOAc. The extract thus acquired was washed with 20 mL of brine thrice. The organic layer was dried over MgSO₄ followed by a concentration process under reduced pressure. The resultant brownish-black thick mass was subjected to a purification treatment employing silica gel column chromatography (EtOAc-hexanes (3:1) as a mobile phase), thereby obtaining 3-[6-M ethoxy-4′-(tetrahydro-pyran-2-yloxy)-biphenyl-3-yl]-propane-1,2-diol (compound 5) as a colorless thick gum (99% yield).

### Detected Properties of the Title Compound:

¹H NMR (400 MHz, CDCl₃, δ): 7.44 (d, J=8.7 Hz, 2H), 7.11 (m, 4H), 6.90 (d, J=8.2 Hz, 1H), 5.46 (t, J=3.2 Hz, 1H), 3.93 (m, 2H), 3.78 (s, 3H), 3.71 (m, 1H), 3.61 (m, 1H), 3.52 (m, 1H), 2.74 (m, 2H), 2.30 (brs, 2H), 2.05 (m, 1H), 1.87 (m, 2H), 1.66 (m, 3H). ¹³C NMR (100.6 MHz, CDCl₃, δ): 156.2, 155.2, 131.6, 131.5, 130.5, 129.8, 128.9, 116.0, 111.4, 96.4, 73.2, 66.0, 62.1, 55.7, 38.9, 30.4, 25.3, 18.8. IR (neat): 3367, 1634, 1365, 1216, 927 cm⁻¹. EIMS (70 eV) m/z: 358.4 (M⁺, 2.4), 213 (100), 274.3 (69.8), 197.2 (12.9), 85.1 (16.8). HRMS-EI (m/z): M⁺ calc. for C₂₁H₂₆O₅: 358.1780. found: 358.1768.

### Step III. Preparation of 5′-allyl-2′-methoxy-biphenyl-4-ol (Compound 6)

3.24 g of the 3-[6-methoxy-4′-(tetrahydro-pyran-2-yloxy)-biphenyl-3-yl]-propane-1,2-diol (compound 5) as obtained in Step II of this example was dissolved in 50 mL of dry dichloromethane (DCM). 4.5 mL of triethylamine (TEA) was added into the resultant solution under argon, followed by a cooling process in an ice bath for 30 minutes. 2.13 mL of methanesulfonyl chloride was added slowly into the cooled mixture over a period of 5 minutes. Stirring was conducted at room temperature for 2 hours (TLC monitoring). 50 mL of DCM was added. The resultant organic layer was repeatedly washed with a saturated NaHCO₃ solution (2×20 mL) and brine (3×20 mL), followed by a drying process using MgSO₄ and a concentration process under vacuo. The crude product thus obtained was dissolved in 50 mL of dry dimethylformamide (DMF), followed by adding 5.48 g of NaI and 8.84 g of zinc dust under argon. Heating was performed at 140° C. for 18 hours (TLC monitoring). A filtration treatment was then conduced using celite so as to remove all solids, followed by washing with EtOAc. A concentration process under vacuum was performed so as to remove DMF. The resultant concentrated product was poured into water, followed by an extraction treatment with EtOAc (2×50 mL). The resultant organic layer was washed with brine (3×30 mL) and dried over MgSO₄. A concentration process under vacuum was then conducted such that a crude product was obtained. The crude product was subjected to a purification treatment employing silica gel column chromatography (EtOAc-hexane (3:22) as a mobile phase), thereby obtaining 5′-allyl-2′-methoxy-biphenyl-4-ol (compound 6) as a colourless oil (85% yield).

### Detected Properties of the Title Compound:

¹H NMR (400 MHz, CDCl₃, δ): 7.43 (d, J=7.2 Hz, 2H), 7.12 (m, 2H), 6.91 (m, 1H, merged with a doublet), 6.89 (d, J=7.2 Hz, 2H merged with a multiplet), 6.01 (m, merged with brs, 2H), 5.10 (m, 2H), 3.80 (s, 3H), 3.39 (d, J=6.7 Hz, 2H). ¹³C NMR (100.6 MHz, CDCl₃, δ): 154.9, 154.8, 137.8, 132.3, 130.9, 130.9, 130.8, 130.3, 128.0, 115.6, 115.0, 111.4, 55.7, 39.5. IR (neat): 3379, 3016, 2950, 2840, 1736, 1604, 1511, 1492, 1462, 1440, 1407, 1358, 1253, 1229, 1174, 1042, 831, 812, 765, 639 cm⁻¹. EIMS (70 eV) m/z: 240.2 (M⁺, 100), 184.1 (36.84), 197.1 (18.75). HRMS-EI (m/z): M⁺ calc. for C₁₆H₁₆O₂: 240.1150. found: 240.1142.

### Step IV. Preparation of 5-allyl-4′-allyloxy-2-methoxy-biphenyl (Compound 7)

4.0 g of the 5′-allyl-2′-methoxy-biphenyl-4-ol (compound 6) as obtained in Step III of this example was dissolved in 120 mL of acetone. 5.0 g of K₂CO₃ and 2.12 mL of allyl bromide were successively added to the resultant solution under argon. The resultant mixture was stirred at room temperature overnight (TLC monitoring). An evaporation process under vacuum was conducted. The residue thus acquired was subjected to an extraction treatment with 100 mL of EtOAc, followed by washing with brine (3×10 mL). The organic layer was dried over anhydrous MgSO₄. A concentration process under vacuum was conducted such that an extract was obtained. The extract was subjected to a purification treatment employing silica gel column chromatography (EtOAc-hexane (1:19) as a mobile phase), such that 5-allyl-4′-allyloxy-2-methoxy-biphenyl (compound 7) as a colorless oil was obtained (92% yield).

### Detected Properties of the Title Compound:

¹H NMR (400 MHz, CDCl₃, δ): 7.47 (d, J=8.3 Hz, 2H), 7.12 (m, 2H), 6.97 (d, J=8.8 Hz, 2H), 6.92 (d, J=8.2 Hz, 1H), 6.05 (m, 2H), 5.45 (d, J=17.2 Hz, 1H), 5.31 (d, J=10.5 Hz, 1H), 5.11 (d, J=17.6 Hz, 1H), 5.07 (d, J=10.3 Hz, 1H), 4.59 (d, J=5.4 Hz, 2H), 3.80 (s, 3H), 3.38 (d, J=6.7 Hz, 2H). ¹³C NMR (100.6 MHz, CDCl₃, δ): 157.7, 154.9, 137.8, 133.5, 132.3, 131.1, 130.9, 130.6, 130.3, 128.0, 117.6, 115.6, 114.3, 111.4, 68.9, 55.7, 39.4. IR (neat): 3065, 2994, 2895, 2835, 1637, 1607, 1577, 1506, 1495, 1465, 1437, 1421, 1399, 1262, 1234, 1176, 1146, 1108, 1039, 1023, 993, 919, 831, 809, 762, 636 cm⁻¹. EIMS (70 eV) m/z: 280.2 (M⁺, 91.6), 239.2 (100), 169.1 (14.0). HRMS-EI (m/z): M⁺ calc. for C₁₉H₂₀O₂: 280.1463. found: 280.1465.

### Step V. Preparation of 3,5′-diallyl-2′-methoxy-biphenyl-4-ol (Compound 8)

4.0 g of the 5-allyl-4′-allyloxy-2-methoxy-biphenyl (compound 7) as obtained in Step IV of this example was dissolved in 100 mL of dry hexane. To the resultant solution was slowly added 35 mL of a 1 M solution of diethyl aluminum chloride (Et₂AlCl) in hexane under argon. Stirring was conducted at room temperature for 2 hours (TLC monitoring). The resultant reaction mixture was cooled to 0° C., and the reaction was quenched via cautious addition of a 1 N HCl solution. An extraction treatment using 100 mL of EtOAc was conducted. The resultant organic layer was washed thoroughly with brine (3×25 mL). The thus obtained extract was dried over MgSO₄, followed by a concentration process under reduced pressure. Accordingly, 3,5′-diallyl-2′-methoxy-biphenyl-4-ol (compound 8) as a colorless oily mass was acquired (100% yield).

### Detected Properties of the Title Compound:

¹H NMR (400 MHz, CDCl₃, δ): 7.31 (dd, J=2.2, 8.2 Hz, 1H), 7.27 (d, J=2.2 Hz, 1H), 7.10 (s, 1H), 7.09 (d, J=2.3 Hz, 1H), 6.89 (d, J=8.4 Hz, 1H), 6.85 (d, J=8.3 Hz, 1H), 6.01 (m, 2H), 5.13 (m, 4H), 4.97 (d, 1H), 3.78 (s, 3H), 3.46 (d, J=6.4 Hz, 2H), 3.37 (d, J=6.7 Hz, 2H). ¹³C NMR (100.6 MHz, CDCl₃, δ): 154.9, 153.3, 137.9, 136.6, 132.4, 131.6, 131.2, 131.1, 130.4, 129.1, 128.1, 125.0, 116.6, 115.7, 115.5, 114.4, 55.8, 39.4, 35.3. IR (neat): 3434, 3076, 3000, 2906, 2829, 1739, 1637, 1607, 1492, 1462, 1432, 1267, 1231, 1179, 1116, 1026, 998, 916, 814, 644 cm⁻¹. EIMS (70 eV) m/z: 280.3 (M⁺, 91.6), 224.2 (35.9), 197.1 (9.1). HRMS-EI (m/z): M⁺ calc. for C₁₉H₂₀O₂: 280.1463. found: 280.1464.

### Step VI. Formation of Product 1 (Compound 1)

500 mg of the 3,5′-diallyl-2′-methoxy-biphenyl-4-ol (compound 8) as obtained in Step V of this example was dissolved into 5 mL of DCM. 3.8 mL of a 1 M solution of BBr₃ in dry DCM was added to the resultant solution over a period of 5 minutes under argon. The resultant mixture was stirred at ambient temperature for 25 minutes (TLC monitoring). The reaction was quenched with a saturated NaHCO₃ solution. An extraction treatment with DCM (3×20 mL) was conducted. The resultant organic layer was washed with brine (2×20 mL), and was dried over MgSO₄, followed by a concentration process under reduced pressure. A gray solid residue was formed and was subjected to a purification treatment employing silica gel column chromatography (EtOAc-hexane (15:85) as a mobile phase). Therefore, 5,3′-diallyl-biphenyl-2,4′-diol (product 1 or compound 1) as a white solid was obtained (90% yield).

### Detected Properties of the Title Compound:

M.p.: 84-86° C. ¹H NMR (400 MHz, CDCl₃, δ): 7.23 (m, 2H), 7.06 (m, 2H), 6.91 (m, 2H), 6.02 (m, 2H), 5.32 (b, 1H), 5.20 (m, 3H), 5.09 (m, 2H), 3.47 (d, J=6.4 Hz, 2H), 3.36 (d, J=6.7 Hz, 2H). ¹³C NMR (100.6 MHz, CDCl₃, δ): 153.9, 150.8, 137.8, 136.0, 132.3, 131.2, 130.3, 129.6, 128.8, 128.6, 127.8, 126.5, 116.9, 116.6, 115.6, 39.4, 35.1. EIMS (70 eV) m/z: 266.3 (M⁺, 100), 237.2 (13.1), 197.1 (9.6) 184.1 (10.8). HRMS-EI (m/z): M⁺ calc. for C₁₈H₁₈O₂: 266.1307. found: 266.1311.

### Synthesis Ex. 2

### 3′-Allyl-5-propyl-biphenyl-2,4′-diol (Product 2 or Compound 13)

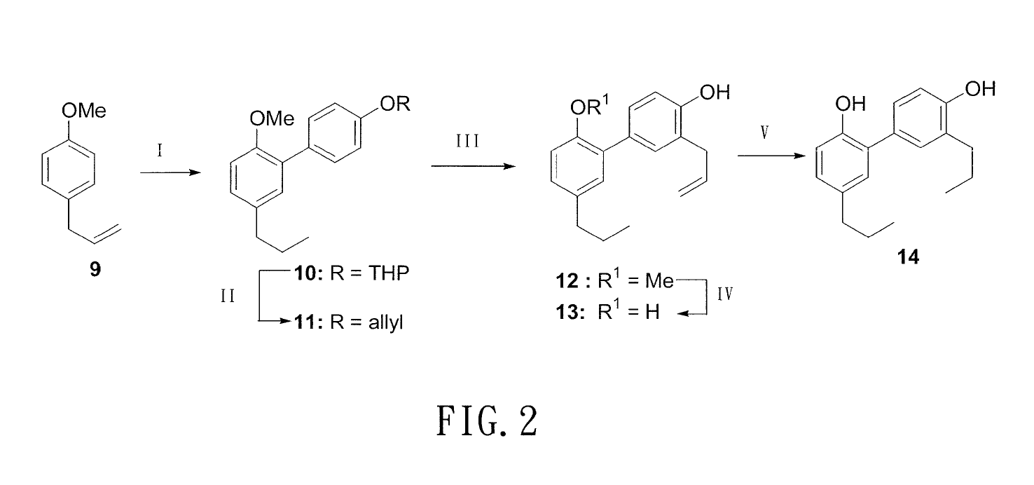

Synthesis of product 2 is outlined in FIG. 2.

### Step I. Preparation of 2-(2′-methoxy-5′-propyl-biphenyl-4-yloxy)-tetrahydro-pyran (Compound 10)

100 mg of palladium on carbon (10 wt % of palladium) was added into 5 mL of methanol (MeOH) so as to prepare a suspension of palladium on carbon. 1.0 g of 4-allylanisole (compound 9) (Aldrich) was dissolved in 10 mL of MeOH so as to form a solution of 4-allylanisole. The solution of 4-allylanisole was mixed with the suspension of palladium on carbon, followed by stirring under a hydrogen pressure of 60 psi for 2 hours (TLC monitoring). The resultant mixture was subjected to a filtration treatment so as to remove all of the solids therein. Subsequently, an evaporation process was conducted such that a crude residue was obtained.

1.0 g of the crude residue was dissolved in 10 mL of acetic acid (AcOH). To the resultant solution was slowly added 0.5 mL of bromine.

The resultant mixture was stirred at ambient temperature for 18 hours (TLC monitoring). An evaporation process under vacuum was performed, followed by an extraction treatment with 20 mL of EtOAc. The extract was washed thoroughly with a saturated Na₂S₂O₃ solution (10 mL), a NaHCO₃ solution (10 mL), and brine (3×10 mL). The organic layer was subjected to a concentration process under reduced pressure such that a concentrated product was obtained (1.2 g, 78% yield).

500 mg of the concentrated product and 800 mg of 4-(tetrahydro-2H-pyran-2-yloxy)-phenylboronic acid pinacol ester (compound 4) were added in 8 mL of DME. To the resultant solution was added 89 mg of the catalyst, PdCl₂(dppf).CH₂Cl₂, under argon, followed by adding 3 mL of an aqueous Na₂CO₃ solution (2 M). The resultant mixture was stirred at 80° C. for 18 hours. An evaporation process was conducted. A dark residue was formed and was then subjected to an extraction treatment using EtOAc. The organic layer was repeatedly washed with brine (3×20 mL), and was subjected to a concentration process under reduced pressure. The resultant dark brown oil was subjected to a purification treatment employing silica gel column chromatography (EtOAc-hexane (1:19) as a mobile phase). Therefore, 2-(2′-methoxy-5′-propyl-biphenyl-4-yloxy)-tetrahydro-pyran (compound 10) as a gum was obtained (83% yield).

### Detected Properties of the Title Compound:

¹H NMR (400 MHz, CDCl₃, δ): 7.51 (d, J=2.1 Hz, 2H), 7.30 (m, 4H merged with doublet), 6.91 (d, J=8.3 Hz, 1H), 5.49 (t, J=3.2 Hz, 1H), 3.94 (m, 1H), 3.81 (s, 3H), 3.62 (m, 1H), 2.60 (t, J=7.4 Hz, 2H), 2.05 (m, 1H), 1.91 (m, 2H), 1.72-1.65 (m, 5H, merged with a quatrate), 0.99 (t, J=7.4 Hz, 3H). ¹³C NMR (100.6 MHz, CDCl₃, δ): 156.2, 154.6, 134.9, 132.0, 130.9, 130.5, 130.0, 127.9, 111.2, 96.4, 62.0, 55.7, 37.3, 30.5, 25.3, 24.9, 18.8, 13.9. IR (neat): 2952, 2871, 1608, 1513, 1492, 1463, 1454, 1440, 1402, 1384, 1355, 1263 cm⁻¹. EIMS (70 eV) m/z: 326.3 (1.1), 242.2 (100), 213.2 (54.4), 197.1 (5.2), 170.1 (3.2), 141.1 (2.6), 115.1 (3.5), 85.1 (8.8). HRMS-EI (m/z): M⁺ calc. for C₂₁H₂₆O₃: 326.1882. found: 326.1885.

### Step II. Preparation of 4′-allyloxy-2-methoxy-5-propyl-biphenyl (Compound 11)

300 mg of the 2-(2′-methoxy-5′-propyl-biphenyl-4-yloxy)-tetrahydro-pyran (compound 10) as obtained in Step I of this example was dissolved in 25 mL of MeOH. A catalytic amount of p-toluenesulfonic acid monohydrate (PTSA) was added into the resultant solution, followed by stirring at ambient temperature for 5 hours (TLC monitoring). The resultant mixture was subjected to an evaporation process under reduced pressure. The thus formed residue was subjected to an extraction treatment using 15 mL of EtOAc. The organic layer was washed with a saturated NaHCO₃ solution (10 mL) and brine (2×10 mL), followed by a concentration process under reduced pressure. 220 mg of the resultant concentrated product was subjected to an allylation reaction generally according to the method as described in Step IV of Synthesis Example 1. The thus obtained crude product was subjected to a purification treatment with silica gel column chromatography (EtOAc-hexane (1:9) as a mobile phase). Thus, 4′-allyloxy-2-methoxy-5-propyl-biphenyl (compound 11) as an oil was obtained (93% yield).

### Detected Properties of the Title Compound:

¹H NMR (400 MHz, CDCl₃, δ): 7.50 (d, J=8.7 Hz, 2H), 7.13 (m, 2H), 6.99 (d, J=8.7 Hz, 2H), 6.91 (d, J=8.2 Hz, 1H), 6.11 (m, 1H), 5.47 (d, J=17.2 Hz, 1H), 5.33 (d, J=10.5 Hz, 1H), 4.60 (d, J=3.8 Hz, 2H), 3.81 (s, 3H), 2.59 (t, J=7.4 Hz, 2H), 1.66 (m, 2H), 0.98 (t, J=7.4 Hz, 3H). ¹³C NMR (100.6 MHz, CDCl₃, δ): 157.7, 154.6, 134.9, 133.5, 131.3, 130.9, 130.6, 129.9, 127.9, 117.7, 114.3, 111.2, 68.9, 55.7, 37.2, 24.8, 13.9. IR (neat): 2956, 2629, 2869, 2832, 1608, 1573, 1515, 1494, 1463, 1442, 1423, 1402 cm⁻¹. EIMS (70 eV) m/z: 282.3 (M⁺, 73.4), 241.2 (100), 212.2 (43.6), 170.2 (54.2), 141.1 (13.0), 115.1 (17.8), 57.1 (21.7). HRMS-EI (m/z): M⁺ calc. for C₁₉H₂₂O₂: 282.1620. found: 282.1614.

### Step III. Preparation of 3-allyl-2′-methoxy-5′-propyl-biphenyl-4-ol (Compound 12)

180 mg of the 4′-allyloxy-2-methoxy-5-propyl-biphenyl (compound 11) as obtained in the Step II of this example was subjected to a rearrangement reaction generally according to the method described in Step V of Synthesis Example 1. Thus, 3-allyl-2′-methoxy-5′-propyl-biphenyl-4-ol (compound 12) as an oil was formed (99% yield).

### Detected Properties of the Title Compound:

¹H NMR (400 MHz, CDCl₃, δ): 7.33 (m, 2H), 7.11 (m, 2H), 6.90 (d, J=8.2 Hz, 1H), 6.85 (d, J=8.2 Hz, 1H), 6.08 (m, 1H), 5.19 (m, 3H), 3.80 (s, 3H), 3.48 (d, J=6.4 Hz, 2H), 2.59 (t, J=7.4 Hz, 2H), 1.67 (m, 2H), 0.98 (t, J=7.4 Hz, 3H). ¹³C NMR (100.6 MHz, CDCl₃, δ): 154.5, 153.3, 136.6, 135.0, 131.5, 131.4, 130.9, 130.1, 129.1, 127.8, 124.8, 116.5, 115.5, 111.2, 55.8, 37.3, 35.4, 24.8, 13.9. IR (neat): 3457, 3075, 3002, 2958, 2929, 2869, 2834, 1637, 1608, 1508, 1490, 1463 cm⁻¹. EIMS (70 eV) m/z: 282.3 (M⁺, 100), 253.3 (68.2), 197.2 (25.4), 165.1 (7.5), 105.1 (8.3), 55.1 (3.6). HRMS-EI (m/z): M⁺ calc. for C₁₉H₂₂O₂: 282.1620. found: 282.1616.

### Step IV. Formation of Product 2 (Compound 13)

180 mg of the 3-allyl-2′-methoxy-5′-propyl-biphenyl-4-ol (compound 12) as obtained in Step III of this example was subjected to a demethylation reaction using BBr₃ generally according to the method described in Step VI of Synthesis Example 1. The resultant crude product was subjected to a purification treatment employing silica gel column chromatography (EtOAc-hexane (3:17) as a mobile phase). Accordingly, 3′-allyl-5-propyl-biphenyl-2,4′-diol (product 2 or compound 13) as a solid was obtained (88% yield).

### Detected Properties of the Title Compound:

M.p.: 103-104° C. ¹H NMR (400 MHz, CDCl₃, δ): 7.23 (m, 2H), 7.03 (m, 2H), 6.89 (m, 2H), 6.04 (m, 1H), 5.30 (brs, 1H), 5.19 (m, 3H), 3.46 (d, J=6.4 Hz, 2H), 2.54 (t, J=7.4 Hz, 2H), 1.63 (m, 2H), 0.95 (t, J=7.4 Hz, 3H). ¹³C NMR (100.6 MHz, CDCl₃, δ): 153.9, 150.4, 136.0, 134.9, 131.2, 130.1, 129.8, 128.7, 128.6, 127.5, 126.4, 116.9, 116.5, 115.4, 37.2, 35.2, 24.9, 13.9. IR (neat): 3374, 3079, 3025, 2958, 2927, 2871, 1639, 1608, 1587, 1506, 1492, 1463 cm⁻¹. EIMS (70 eV) m/z: 268.3 (M⁺, 100), 239.2 (96.0), 197.2 (21.8), 181.2 (8.3), 165.1 (7.0), 98.1 (7.4). HRMS-EI (m/z): M⁺ calc. for C₁₈H₂₀O₂: 268.1463. found: 268.1465.

### Synthesis Ex. 3

### 5,3′-Dipropyl-biphenyl-2,4′-diol (Product 3 or Compound 14)

Synthesis of product 3 is outlined in FIG. 2 as well (Step V).

75 mg of product 2 (compound 13) as obtained in Synthesis Example 2 was dissolved in 10 mL of MeOH. To the resultant solution was added 10 mg of palladium on carbon (10 wt % palladium), which was previously wetted with MeOH. The resulting mixture was stirred at ambient temperature under a hydrogen pressure of 60 psi for 2 hours, followed by a filtration treatment using celite. The filtrate was subjected to a concentration process under vacuum. Consequently, 5,3′-dipropyl-biphenyl-2,4′-diol (product 3 or compound 14) as a solid was obtained (99% yield).

### Detected Properties of the Title Compound:

M.p.: 98-99° C. ¹H NMR (400 MHz, CDCl₃, δ): 7.24 (d, J=2.2 Hz, 1H), 7.19-7.16 (m, 1H), 7.07-7.03 (m, 2H), 6.94 (d, J=8.0 Hz, 1H), 6.85 (d, J=8.0 Hz, 1H), 5.30-5.17 (m, 2H), 2.64 (t, J=7.5 Hz, 2H), 2.56 (t, J=7.8 Hz, 2H), 1.74-1.60 (m, 4H), 1.03 (m, 6H). ¹³C NMR (100.6 MHz, CDCl₃, δ): 153.3, 150.3, 135.0, 131.0, 130.1, 129.5, 129.53, 128.6, 128.6, 127.73, 127.7, 115.9, 115.4, 37.3, 32.1, 24.9, 22.9, 14.1, 13.9. IR (neat): 3350, 2925, 1736, 1504, 1429, 1375, 1234, 1125, 823 cm⁻¹. EIMS (70 eV) m/z: 270.3 (M⁺, 73), 241.2 (100), 199.2 (24.5), 181.2 (5.1), 165.1 (4.3), 106.1 (9.0). HRMS-EI (m/z): M⁺ calc. for C₁₈H₂₂O₂: 270.1620. found: 270.1614.

### Synthesis Ex. 4

### 5-Allyl-3′-propyl-biphenyl-2,4′-diol (Product 4 or Compound 19)

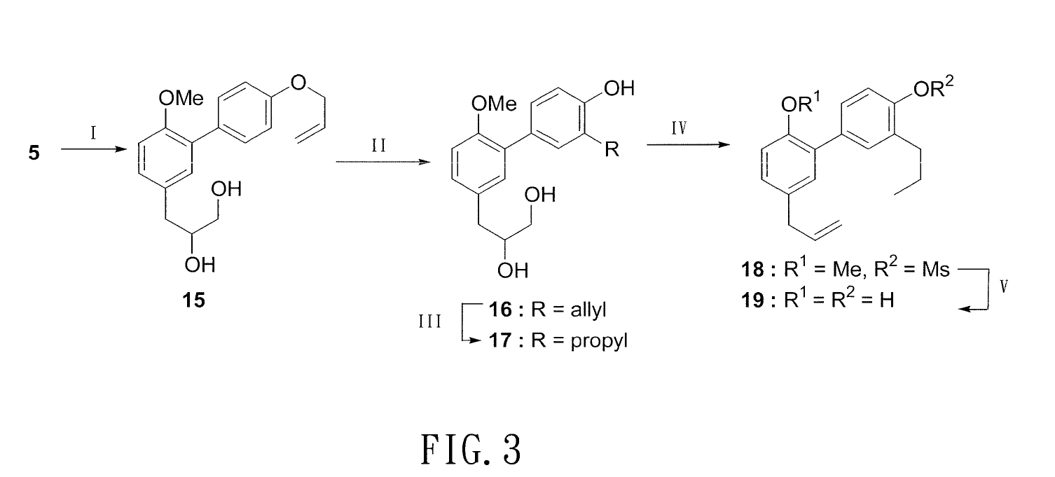

Synthesis of product 4 is outlined in FIG. 3.

### Step I. Preparation of 3-(4′-allyloxy-6-methoxy-biphenyl-3-yl)-propane-1,2-diol (Compound 15)

785 mg of 3-[6-Methoxy-4′-(tetrahydro-pyran-2-yloxy)-biphenyl-3-yl]-propane-1,2-diol (compound 5), which can be prepared using the method as described in Steps I-II of Synthesis Example 1, was sequentially subjected to a PTSA-catalyzed THP (tetrahydropyran) deprotection reaction and an allyation reaction (at room temperature for 36 hours) generally according to the method as described in Step II of Synthesis Example 2. The resultant crude product was subjected to a purification treatment using silica gel column chromatography (EtOAc-hexane (1:1) as a mobile phase). Therefore, 3-(4′-allyloxy-6-methoxy-biphenyl-3-yl)-propane-1,2-diol (compound 15) as a solid was obtained (89% yield over two steps).

### Detected Properties of the Title Compound:

M.p.: 50-52° C. ¹H NMR (400 MHz, CDCl₃, δ): 7.44 (m, 2H), 7.12 (m, 2H), 6.95 (m, 2H), 6.90 (d, J=8.2 Hz, 1H), 6.07 (m, 1H), 5.43 (dd, J=1.52, 17.2 Hz, 1H), 5.29 (dd, J=1.3, 10.5 Hz, 1H), 4.56 (m, 2H), 3.91 (m, 1H), 3.78 (s, 3H), 3.68 (dd, J=3.1, 11.1 Hz, 1H), 3.51 (dd, J=7, 11.1 Hz, 1H), 2.73 (m, 2H), 2.24 (brs, 2H). ¹³C NMR (100.6 MHz, CDCl₃, δ): 157.8, 155.3, 133.4, 131.6, 130.8, 130.6, 130.4, 129.8, 128.9, 117.7, 114.3, 111.4, 73.2, 68.9, 66.1, 55.7, 38.9. IR (KBr): 3367, 923, 1610, 1509, 1243, 1172, 1025, 821, 752 cm⁻¹. EIMS (70 eV) m/z: 314.2 (M⁺, 100), 273.1 (88.0), 253.1 (82.8), 212.1 (51.5), 197.1 (32.1), 169.1 (34.1)), 152.1 (32.1), 141.1 (37.9), 115.0 (56.7), 61.0 (23.5). HRMS-EI (m/z): M⁺ calc. for C₁₉H₂₂O₄: 314.1518. found: 314.1513.

### Step II. Preparation of 3-(3′-allyl-4′-hydroxy-6-methoxy-biphenyl-3-yl)-propane-1,2-diol (Compound 16)

315 mg of the 3-(4′-allyloxy-6-methoxy-biphenyl-3-yl)-propane-1,2-dial (compound 15) as obtained in Step I of this example was dissolved in 10 mL of dry DCM. To the resultant solution was added 0.45 mL of a 1 M solution of Et₂AlCl in hexane, followed by stirring at ambient temperature for 2 hours. The reaction was carefully quenched by adding 3 mL of 1 N HCl at 0° C. A dilution process with 10 mL of DCM was then conducted. The diluent was washed with brine (3×10 mL) and dried over MgSO₄. Subsequently, an evaporation process under reduced pressure was performed. Accordingly, 3-(3′-allyl-4′-hydroxy-6-methoxy-biphenyl-3-yl)-propane-1,2-diol (compound 16) as an oil was obtained (98% yield).

### Detected Properties of the Title Compound:

¹H NMR (400 MHz, CDCl₃, δ): 7.26 (m, 2H), 7.11 (m, 2H), 6.89 (d, J=8.1 Hz, 1H), 6.80 (d, J=8.8 Hz, 1H), 6.03 (m, 1H), 5.80 (brs, 1H), 5.16 (m, 2H), 3.94 (m, 1H), 3.79 (s, 3H), 3.70 (dd, J=3.0, 11.2 Hz, 1H), 3.53 (dd, J=7.0, 11.2 Hz, 1H), 3.43 (d, J=6.4 Hz, 2H), 2.73 (m, 2H), 2.34 (brs, 2H). ¹³C NMR (100.6 MHz, CDCl₃, δ): 155.3, 153.4, 136.6, 131.6, 130.7, 130.6, 129.7, 128.9, 128.8, 125.2, 116.4, 115.4, 111.5, 73.2, 66.0, 55.7, 38.9, 35.1. IR (neat): 3353, 2931, 1623, 1496, 1440, 1251, 1081, 1025, 906, 821, 736 cm⁻¹. EIMS (70 eV) m/z: 314.2 (M⁺, 64.9), 253.2 (100), 213.1 (7.5), 197.1 (16.9), 165.1 (4.6). HRMS-EI (m/z): M⁺ calc. for C₁₉H₂₂O₄: 314.1518. found: 314.1514.

### Step III. Preparation of 3-(4′-hydroxy-6-methoxy-3′-propyl-biphenyl-3-yl)-propane-1,2-diol (Compound 17)

250 mg of the 3-(3′-allyl-4′-hydroxy-6-methoxy-biphenyl-3-yl)-propane-1,2-diol (compound 16) as obtained in Step II of this example was subjected to a hydrogenation reaction generally according to the method described in Synthesis Example 3, thereby forming 3-(4′-hydroxy-6-methoxy-3′-propyl-biphenyl-3-yl)-propane-1,2-diol (compound 17) as an oil (100% yield).

### Detected Properties of the Title Compound:

¹H NMR (400 MHz, CDCl₃, δ): 7.26 (t, J=2.1 Hz, 1H), 7.17 (dd, J=2.1, 8.2 Hz, 1H), 7.09 (m, 2H), 6.86 (d, J=8.2 Hz, 1H), 6.72 (d, J=8.2 Hz, 1H), 6.12 (d, 1H), 3.93 (m, 1H), 3.76 (s, 3H), 3.68 (dd, J=11.3, 2.8 Hz, 1H), 3.51 (dd, J=7.1, 11.3 Hz, 1H), 2.71 (m, 2H), 2.59 (t, J=7.4 Hz, 2H), 1.64 (m, 2H), 0.97 (t, J=7.4 Hz, 3H). ¹³C NMR (100.6 MHz, CDCl₃, δ): 155.2, 153.0, 131.6, 131.4, 130.8, 130.3, 129.7, 128.7, 128.2, 128.0, 114.9, 111.5, 73.3, 65.9, 55.7, 38.8, 32.2, 22.9, 14.1. IR (neat): 3367, 2931, 2875, 1610, 1490, 1427, 1251, 1124, 1018, 906, 815, 736 cm⁻¹. EIMS (70 eV) m/z: 316.2 (M⁺, 56.7), 255.2 (100), 225.1 (4.8), 213.1 (10.5), 197.1 (11.8), 165.1 (3.4). HRMS-EI (m/z): M⁺ calc. for C₁₉H₂₄O₄: 316.1675. found: 316.1681.

### Step IV. Preparation of Methanesulfonic Acid 5′-allyl-2′-methoxy-3-propyl-biphenyl-4-yl ester (Compound 18)

240 mg of the 3-(4′-hydroxy-6-methoxy-3′-propyl-biphenyl-3-yl)-propane-1,2-diol (compound 17) as obtained in Step III of this example was dissolved in 10 mL of dichloromethane. 0.52 mL of TEA and 0.23 mL of methanesulfonyl chloride (MsCl) were successively added into the resultant solution at 0° C. The mixture thus obtained was stirred at room temperature for 2 hours. The resultant organic layer was diluted with 20 mL of dichloromethane, followed by washing with a saturated NaHCO₃ solution (2×10 mL) and brine (3×10 mL), and drying with MgSO₄. An evaporation process under vacuum was conducted.

The residue thus obtained was dissolved in 5 mL of DMF, followed by adding 436 mg of NaI and 436 mg of Zn. The thus formed mixture was heated at 140° C. with stirring under argon for 2 hours, followed by dilution with 10 mL of EtOAc (10 mL). A filtration treatment was performed so as to remove solids. The resultant filtrate was thoroughly washed with water (5×50 mL) and brine (3×10 mL), followed by drying with MgSO₄. An evaporation process under reduced pressure was conducted, thereby forming a crude product. The crude product was subjected to a purification treatment employing silica gel column chromatography (EtOAc-hexane (1:9) as a mobile phase). Thus, methanesulfonic acid 5′-allyl-2′-methoxy-3-propyl-biphenyl-4-yl ester (compound 18) as a solid was obtained (85% yield over two steps).

### Detected Properties of the Title Compound:

M.p.: 64-66° C. ¹H NMR (400 MHz, CDCl₃, δ): 7.42 (d, J=1.96 Hz, 1H), 7.38 (dd, J=2.1, 8.4 Hz, 1H), 7.33 (d, J=8.4 Hz, 1H), 7.15-7.10 (m, 1H), 5.12-5.06 (m, 2H), 3.79 (s, 3H), 3.37 (d, J=6.7 Hz, 2H), 3.21 (s, 3H), 2.71 (t, J=7.4 Hz, 2H), 1.72-1.66 (m, 2H), 1.00 (t, J=7.4 Hz, 3H). ¹³C NMR (100.6 MHz, CDCl₃, δ): 154.8, 146.6, 137.6, 134.7, 132.4, 132.0, 131.0, 129.4, 128.8, 128.4, 121.3, 115.7, 111.4, 55.7, 39.4, 38.2, 32.2, 23.2, 14.0. IR (neat): 3460, 2961, 2872, 1737, 1638, 1608, 1504, 1486, 1464, 1366, 1265, 1238, 1206, 1171, 1111, 1027, 969, 864, 823 cm⁻¹. EIMS (70 eV) m/z: 360.2 (M⁺, 62.2), 281.2 (100), 253.1 (52.8), 240.1 (40.2), 225.1 (16.3), 211.1 (29.0), 181.1 (14.5), 165.1 (23.8), 152.1 (13.9), 128.1 (7.9), 115.1 (14.0), 79.0 (40.6). HRMS-EI (m/z): M⁺ calc. for C₂₀H₂₄SO₄: 360.1395. found: 360.1397.

### Step V. Formation of Product 4 (Compound 19)

200 mg of the methanesulfonic acid 5′-allyl-2′-methoxy-3-propyl-biphenyl-4-yl ester (compound 18) as obtained in Step IV of this example was subjected to a demethylation reaction generally according to the method described in Step VI of Synthesis Example 1, such that a crude product is formed. The crude product was dissolved in 5 mL of an aqueous dioxane solution (water:dioxane=1:1), followed by adding 5 mL of a 20% aqueous NaOH solution. The resultant mixture was heated at 60° C. for 2 hours. An evaporation process under reduced pressure was conducted. Subsequently, the residual aqueous layer was adjusted to pH 6-7 through addition of 1 M HCl. 10 mL of dichloromethane was added. The thus formed organic layer was repeatedly washed with brine (3×10 mL), followed by drying with MgSO₄. A concentration process under reduced pressure was conducted, thereby forming a crude product. The crude product was subjected to a purification treatment employing silica gel column chromatography (EtOAc-hexane (1:5) as a mobile phase). Consequently, 5-allyl-3′-propyl-biphenyl-2,4′-diol (product 4 or compound 19) as a solid was obtained (87% yield over two steps).

### Detected Properties of the Title Compound:

M.p.: 63-65° C. ¹H NMR (400 MHz, CDCl₃, δ): 7.21 (d, J=2.0 Hz, 1H), 7.16 (dd, J=2.0 Hz, J=8.1 Hz, 1H), 7.07-7.04 (m, 2H), 6.91 (d, J=8.1 Hz, 1H), 6.85 (d, J=8.1 Hz, 1H), 6.03-5.93 (m, 1H), 5.23-5.05 (m, 3H), 3.35 (d, J=6.6 Hz, 2H), 2.63 (t, J=7.5 Hz, 2H), 1.73-1.63 (m, 2H), 1.02 (t, J=7.5 Hz, 3H). ¹³C NMR (100.6 MHz, CDCl₃, δ): 153.4, 150.8, 137.8, 132.2, 131.0, 130.2, 129.5, 129.3, 128.8, 127.9, 127.7, 115.9, 115.6, 115.6, 39.5, 32.1, 22.9, 14.1. IR (neat): 3257, 2958, 2929, 2869, 1738, 1635, 1495, 1468, 1426, 1365, 1217, 1129, 999, 911, 822, 775 cm⁻¹. EIMS (70 eV) m/z: 268.2 (M⁺, 100), 239.1 (58.1), 225.1 (8.4), 197.1 (10.1), 165.1 (7.5), 152.1 (6.8), 115.0 (7.6), 77.0 (5.0). HRMS-EI (m/z): M⁺ calc. for C₁₈H₂₀O₂: 268.1463. found: 268.1455.

### Synthesis Ex. 5

### 3′-Allyl-5-prop-2-ynyl-biphenyl-2,4′-diol (Product 5 or Compound 25)

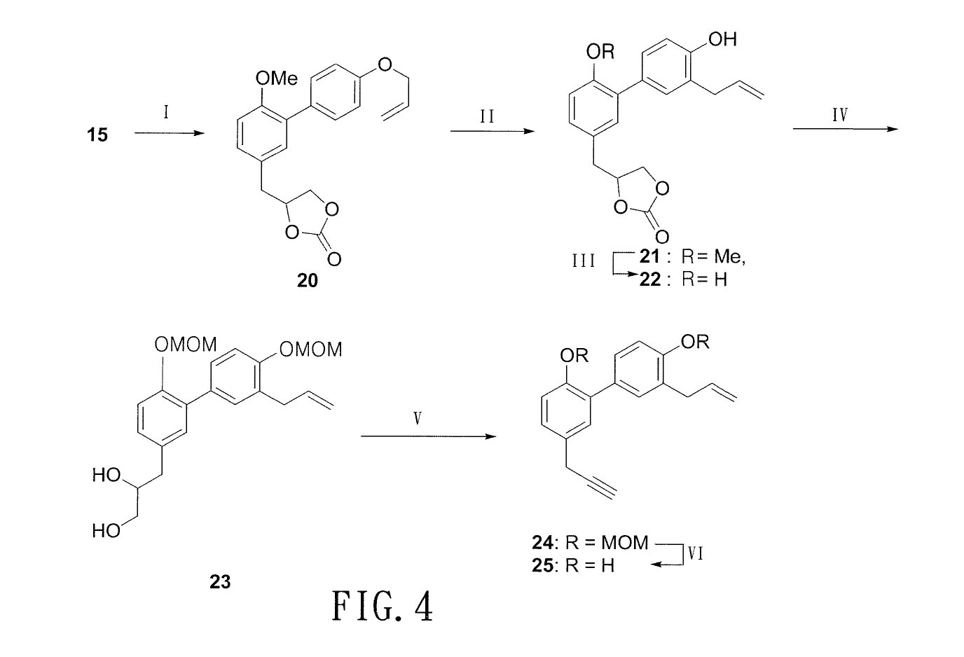

Synthesis of product 5 is outlined in FIG. 4.

### Step I. Preparation of 4-(4′-allyloxy-6-methoxy-biphenyl-3-ylmethyl)-[1,3]dioxolan-2-one (Compound 20)

510 mg of 3-(4′-allyloxy-6-methoxy-biphenyl-3-yl)-propane-1,2-diol (compound 15), which can be prepared using the method as described in Step I of Synthesis Example 4, was dissolved in 20 mL of dichloromethane. 1.5 mL of pyridine was added into the resultant solution under argon. The thus obtained mixture was cooled to −78° C. 1.2 mL of a 20% solution of COCl₂ in toluene was added, followed by stirring at room temperature for 1 hour (TLC monitoring). 10 mL of dichloromethane was added. The resultant organic layer was washed with brine (3×10 mL), followed by drying with MgSO₄. A concentration process under reduced pressure was conducted. Accordingly, 4-(4′-allyloxy-6-methoxy-biphenyl-3-ylmethyl)-[1,3]dioxolan-2-one (compound 20) as an oil was obtained (99% yield).

### Detected Properties of the Title Compound:

¹H NMR (400 MHz, CDCl₃, δ): 7.43 (d, J=8.7 Hz, 2H), 7.12 (m, 2H), 6.96 (d, J=8.7 Hz, 2H), 6.92 (d, J=9.0 Hz, 1H), 6.07 (m, 1H), 5.43 (dd, J=17.2, 1.4 Hz, 1H), 5.30 (dd, J=10.4, 1.2 Hz, 1H), 4.93 (m, 1H), 4.57 (d, J=5.3 Hz, 2H), 4.44 (t, J=8.2 Hz, 1H), 4.19 (dd, J=8.4, 6.9 Hz, 1H), 3.79 (s, 3H), 3.11 (dd, J=14.2, 6.0 Hz, 1H), 2.96 (dd, J=14.2, 6.6 Hz, 1H) ¹³C NMR (100.6 MHz, CDCl₃, δ): 157.9, 155.9, 154.9, 133.3, 131.6, 130.8, 130.5, 130.4, 130.3, 129.0, 125.8, 117.7, 114.4, 111.6, 68.8, 68.4, 55.7, 38.8. IR (neat): 2917, 2837, 1801, 1608, 1515, 1496, 1463, 1395, 1365, 1243, 1175, 1064, 1026, 931, 834 cm⁻¹. EIMS (70 eV) m/z: 340.1 (M⁺, 98.7), 299.1 (100), 253.1 (16.0), 213.1 (26.7), 197.0 (23.1), 169.0 (23.9), 165.0 (33.5), 152.0 (28.1), 141.1 (25.0), 115.0 (43.5), 69.0 (17.3), 55.0 (19.8). HRMS-EI (m/z): M⁺ calc. for C₂₀H₂₀O₅: 340.1311. found: 340.1317.

### Step II. Preparation of 4-(3′-allyl-4′-hydroxy-6-methoxy-biphenyl-3-ylmethyl)-[1,3]dioxolan-2-one (Compound 21)

500 mg of the 4-(4′-Allyloxy-6-methoxy-biphenyl-3-ylmethyl)-[1,3]dioxolan-2-one (compound 20) as obtained in Step I of this example was subjected to a Claisen rearrangement reaction using Et₂AlCl generally according to the method described in Step II of Synthesis Example 4. Thus, 4-(3′-allyl-4′-hydroxy-6-methoxy-biphenyl-3-ylmethyl)-[1,3]dioxolan-2-one (compound 21) as an oil was obtained (98% yield).

### Detected Properties of the Title Compound:

¹H NMR (400 MHz, CDCl₃, δ): 7.28 (dd, J=8.2, 2.1 Hz, 1H), 7.25 (d, J=2.1 Hz, 1H merged with CHCl₃ signal), 7.14-7.11 (m, 2H), 6.92 (d, J=8.0 Hz, 1H), 6.85 (d, J=8.2 Hz, 1H), 6.09-6.02 (m, 1H), 5.24-5.16 (m, 2H), 5.12 (brs, 1H), 4.94-4.91 (m, 1H), 4.44 (t, J=8.2 Hz, 1H), 4.19 (dd, J=8.5, 6.8 Hz, 1H), 3.79 (s, 3H), 3.46 (d, J=6.3 Hz, 2H), 3.11 (dd, J=14.2, 6.0 Hz, 1H), 2.97 (dd, J=14.2, 6.6 Hz, 1H) ¹³C NMR (100.6 MHz, CDCl₃, δ): 155.9, 154.9, 153.6, 136.4, 131.6, 131.45, 130.9, 130.5, 129.0, 128.9, 125.8, 124.9, 116.7, 115.6, 114.4, 111.7, 68.5, 55.7, 38.8, 35.3. IR (neat): 3401, 2920, 1779, 1607, 1492, 1463, 1398, 1365, 1269, 1244, 1177, 1064, 1027, 915, 820, 772 cm⁻¹. EIMS (70 eV) m/z: 340.1 (M⁺, 100), 253.1 (85.2), 237.1 (19.1), 197.0 (37.3), 165.0 (21.5), 152.0 (14.4), 115.0 (13.8), 69.0 (13.5). HRMS-EI (m/z): M⁺ calc. for C₂₀H₂₀O₅: 340.1311. found: 340.1317.

### Step III. Preparation of 4-(3′-allyl-6,4′-dihydroxy-biphenyl-3-ylmethyl)-[1,3]dioxolan-2-one (Compound 22)

470 mg of the 4-(3′-Allyl-4′-hydroxy-6-methoxy-biphenyl-3-ylmethyl)[1,3]dioxolan-2-one (compound 21) as obtained in Step II of this example was subjected to a demethylation reaction using BBr₃ generally according to the method described in Step VI of Synthesis Example 1, thereby forming a crude product. The crude product was subjected to a purification treatment employing silica gel column chromatography (EtOAc-hexane (2:3) as a mobile phase) such that 4-(3′-allyl-6,4′-dihydroxy-biphenyl-3-ylmethyl)-[1,3]dioxolan-2-one (compound 22) as an oil was obtained (96% yield).

### Detected Properties of the Title Compound:

¹H NMR (400 MHz, CDCl₃, δ): 7.19 (m, 2H), 7.06 (m, 2H), 6.92 (m, 2H), 6.02 (m, 1H), 5.46 (brs, 1H), 5.33 (brs, 1H), 5.19 (m, 2H), 4.92 (m, 1H), 4.45 (t, J=8.4 Hz, 1H), 4.19 (dd, J=8.5, 6.8 Hz, 1H), 3.45 (d, J=6.4 Hz, 2H), 3.08 (dd, J=14.2, 6.0 Hz, 1H). 2.95 (dd, J=14.3, 6.5 Hz, 1H). ¹³C NMR (100.6 MHz, CDCl₃, δ): 15.0, 154.3, 152.0, 135.9, 132.2, 131.1, 131.0, 129.6, 128.7, 128.5, 126.6, 125.8, 116.9, 116.7, 116.2, 115.9, 68.5, 38.7, 35.1. IR (neat): 3400, 2970, 1772, 1608, 1488, 1433, 1365, 1229, 1217, 1064 cm⁻¹. EIMS (70 eV) m/z: 326.1 (M⁺, 91.0), 239.1 (100), 197.0 (41.7), 181.0 (23.8), 165.0 (30.5), 152.0 (23.2), 115.0 (32.6), 77.0 (25.1). HRMS-EI (m/z): M⁺ calc. for C₁₉H₁₈O₅: 326.1154. found: 326.1152.

### Step IV. Preparation of 3-(3′-allyl-6,4′-bis-methoxymethoxy-biphenyl-3-yl)-propane-1,2-diol (Compound 23)

A suspension of NaH (132 mg, 60% in parafin) in tetrahydrofuran (THF) (3 mL) was cooled to 0° C. under argon. 450 mg of the 4-(3′-Allyl-6,4′-dihydroxy-biphenyl-3-ylmethyl)-[1,3]dioxolan-2-one (compound 22) as obtained in Step III of this example was added into 3 mL of THF so as to form a solution. The solution was added into the aforesaid suspension, followed by stirring at 0° C. for 30 minutes. To the resultant mixture was added dropwise 0.28 mL of methoxymethylbromide via a syringe. Stirring was conducted for 1 hour. 0.5 mL of water was added, followed by an extraction treatment with 20 mL of EtOAc. The extract was washed with brine (3×10 mL), dried over MgSO₄, and subjected to a concentration process. A crude residue was formed (90% yield).

400 mg of the crude residue was dissolved in 15 mL of ID dioxane-H₂O (1:1). 5 mL of a 20% solution of NaOH in water was added into the resultant solution, followed by heating at 60° C. for 2 hours (TLC monitoring). The thus obtained mixture was cooled to room temperature, and pH thereof was adjusted to 5-6 using 1 M HCl solution. The resultant turbid white residue was taken up in 15 mL of dichloromethane, followed by washing with brine (2×10 mL). After drying and concentration processes, a purification treatment employing silica gel column chromatography (EtOAc-hexane (1:1) as a mobile phase) was conducted. Thus, 3-(3′-Allyl-6,4′-bis-methoxymethoxy-biphenyl-3-yl)-propane-1,2-diol (compound 23) as an oil was obtained (92% yield).

### Detected Properties of the Title Compound:

¹H NMR (400 MHz, CDCl₃, δ): 7.32 (m, 2H), 7.12 (m, merged with CHCl₃ signal, 5H), 6.02 (m, 1H), 5.23 (s, 2H), 5.10 (m, 1H), 5.09 (m, 4H merged with a singlet), 3.90 (m, 1H), 3.70 (m, 1H), 3.55 (m, 1H), 3.51 (s, 3H), 3.44 (m, 2H), 3.40 (s, 3H), 2.75 (m, 2H). ¹³C NMR (100.6 MHz, CDCl₃, δ): 154.1, 152.9, 136.9, 131.7, 131.3, 131.1, 128.9, 128.7, 128.4, 115.8, 115.6, 113.5, 95.0, 94.4, 73.1, 66.1, 56.1, 39.1, 34.5. EIMS (70 eV) m/z: 388.2 (M⁺, 100), 356.1 (36.6), 340.1 (23.1), 311.1 (33.7), 295.1 (62.7), 263.1 (29.3), 251.1 (62.0), 237.1 (46.7), 197.0 (26.0), 165.0 (29.1), 152.0 (18.5), 131.0 (18.0). HRMS-EI (m/z): M⁺ calc, for C₂₂H₂₈O₆: 388.1886. found: 388.1883.

### Step V. Preparation of 3′-allyl-2,4′bis-methoxymethoxy-5-prop-2-ynyl-biphenyl (Compound 24)

340 mg of the 3-(3′-allyl-6,4′-bis-methoxymethoxy-biphenyl-3-yl)-propane-1,2-diol (compound 23) as obtained in Step IV of this example was dissolved in 15 mL of MeOH. The resultant solution was placed in an ice bath, followed by dropwise adding a saturated aqueous solution of NaIO₄ (280 mg). Stirring was conducted for 45 minutes (TLC monitoring). The resultant mixture was subjected to a filtration treatment with celite to remove solids. The thus obtained filtrate was subjected to an evaporation process such that a residue was formed. The residue was subjected to an extraction treatment using 15 mL of EtOAc. The extract was washed with brine, followed by a concentration process under reduced pressure. Accordingly, a thick gummy residue (85% yield) was obtained.

The thick gummy residue was dried under vacuum. 265 mg of the vacuum dried residue was dissolved in 3 mL of dichloromethane. The resultant solution was slowly added to a cold orange suspension of CBr₄ (724 mg), PPh₃ (1.15 g), and K₂CO₃ (302 mg) in dichloromethane (7 mL) at a temperature below 10° C. under argon, followed by stirring at a temperature below 10° C. for 15 minutes (TLC monitoring). The resultant mixture was diluted with 15 mL of dichloromethane, followed by conducting a filtration treatment with a short pad of silica gel so as to remove triphenylphosphine oxide as much as possible. A concentration process was then performed such that a yellow oil was formed (86% yield).

300 mg of the yellow oil was added into 5 mL of THF. To the resultant solution was slowly added 380 μL of a 16% solution of tert-butyllithium (t-BuLi) in pentane at −78° C. The thus obtained mixture was stirred at room temperature for 30 minutes (TLC monitoring). The reaction was quenched using 0.5 mL of H₂O. An extraction treatment with 10 mL of EtOAc was then conducted. The extract was washed with brine, dried, and subjected to a concentration process, thereby forming a residue. The residue was subjected to a purification treatment employing silica gel column chromatography (EtOAc-hexane (1:9) as a mobile phase). Consequently, 3′-Allyl-2,4′-bis-methoxymethoxy-5-prop-2-ynyl-biphenyl (compound 24) as an oil was obtained (91% yield).

### Detected Properties of the Title Compound:

¹H NMR (400 MHz, CDCl₃, δ): 7.34 (m, 2H), 7.26 (m, merged with CHCl₃ signal, 2H), 7.15 (d, J=8.4 Hz, 1H), 7.12 (d, J=9.0 Hz, 1H), 6.03 (m, 1H), 5.24 (s, 2H), 5.11 (m, 1H), 5.09 (s, 2H), 5.06 (m, 1H), 3.59 (d, J=2.7 Hz, 2H), 3.51 (s, 3H), 3.46 (d, J=6.6 Hz, 2H), 3.40 (s, 3H), 2.18 (t, J=2.7 Hz, 1H). ¹³C NMR (100.6 MHz, CDCl₃, δ): 154.1, 152.9, 136.9, 131.7, 131.6, 131.2, 130.4, 129.8, 128.7, 128.4, 127.5, 115.8, 115.6, 113.5, 95.1, 94.4, 82.3, 70.4, 56.1, 34.5, 24.1. IR (neat): 2954, 2827, 2095, 1723, 1683, 1644, 1597, 1488, 1263, 1233, 1200, 1154, 1079, 990, 921 cm⁻¹. EIMS (70 eV) m/z: 352.2 (M⁺, 88.6), 342.2 (49.2), 320.2 (53.5), 289.2 (60.3), 275.2 (100), 261.1 (59.0), 236.1 (80.3), 221.1 (61.7), 189.1 (42.3) 165.1 (66.5), 152.1 (56.3), 139.1 (40.0), 115.1 (40.0), 46.0 (40.3). HRMS-EI (m/z): M⁺ calc. for C₂₂H₂₄O₄: 352.1675. found: 352.1676.

### Step VI. Formation of product 5 (Compound 25)

150 mg of the 3′-allyl-2,4′-bis-methoxymethoxy-5-prop-2-ynyl-biphenyl (compound 24) as obtained in Step V of this example was added into 5 mL of THF. To the resultant solution was added 0.5 mL of a concentrated HCl solution, followed by stirring at room temperature for 5 hours. The resultant mixture was subjected to an evaporation process such that a residue was formed. The residue was neutralized with a NaHCO₃ solution, followed by an extraction treatment with 10 mL of EtOAc. The extract was washed with brine (2×10 mL), dried over MgSO₄, and subjected to a concentration process. The resultant crude residue was subjected to a purification treatment employing silica gel column chromatography (EtOAc-hexane (1:5) as a mobile phase). Therefore, 3′-allyl-5-prop-2-ynyl-biphenyl-2,4′-diol (product 5 or compound 25) as a gum was obtained (92% yield), and was further recrystallized from hexane-diethyl ether (19:1) to become a solid form.

### Detected Properties of the Title Compound:

M.p.: 96° C. ¹H NMR (400 MHz, CDCl₃, δ): 7.21 (m, 4H), 6.92 (m, 2H), 6.04 (m, 1H), 5.18 (m, 4H), 3.56 (d, J=2.0 Hz, 2H), 3.46 (d, J=6.1 Hz, 2H), 2.17 (t, J=2.0 Hz, 1H). ¹³C NMR (100.6 MHz, CDCl₃, δ): 154.1, 151.3, 135.9, 131.2, 129.6, 129.4, 128.6, 128.2, 127.9, 126.4, 117.0, 116.7, 115.8, 82.4, 70.3, 35.2, 23.9. IR (KBr): 3293, 2970, 2095, 1607, 1493, 1365, 1228, 1216, 1119, 821 cm⁻¹. EIMS (70 eV) m/z: 264.1 (M⁺, 100), 235.1 (21.3), 223.1 (13.4), 184.0 (15.7), 165.0 (14.9), 152.0 (10.4), 131.0 (7.5), 115.0 (7.4). HRMS-EI (m/z): M⁺ calc. for C₁₈H₁₆O₂: 264.1150. found: 264.1152.

### Synthesis Ex. 6

### 5-Propyl-3′-prop-2-ynyl-biphenyl-2,4′-diol (Product 6 or Compound 29)

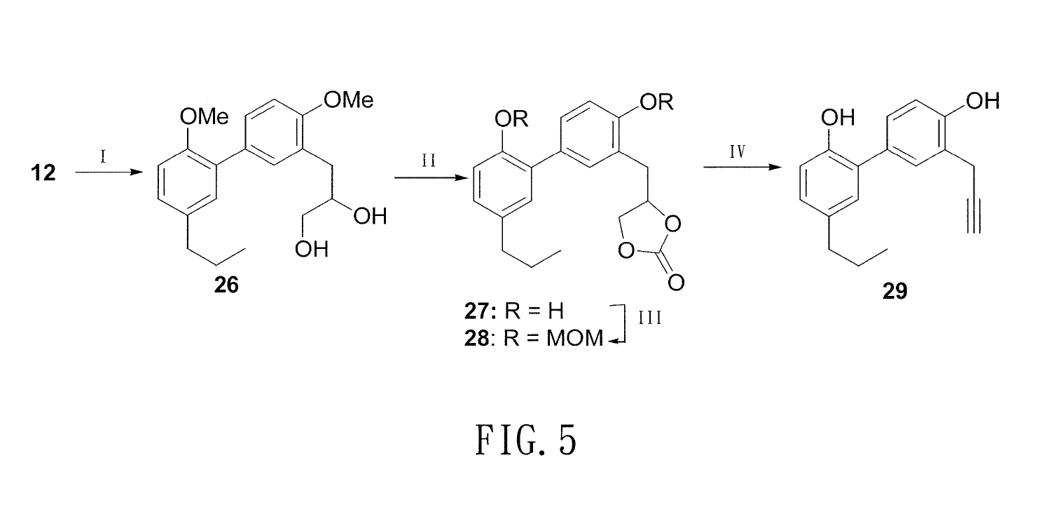

Synthesis of product 6 is outlined in FIG. 5.

### Step I. Preparation of 3-(4,2′-dimethoxy-5′-propyl-biphenyl-3-yl)-propane-1,2-diol (Compound 26)

470 mg of 3-allyl-2′-methoxy-5′-propyl-biphenyl-4-ol (compound 12), which can be prepared using the method as described in Steps I-III of Synthesis Example 2, was added into 10 mL of acetone. 700 mg of K₂CO₃ and 1 mL of MeI were added into the resultant solution, followed by stirring at room temperature overnight. The thus obtained mixture was subjected to an evaporation process such that a residue was formed. The residue was subjected to an extraction treatment with 15 mL of EtOAc. The resultant organic layer was washed with brine (3×10 mL), and dried over MgSO₄. After a concentration process under reduced pressure, a crude product was obtained (97% yield).

Afterwards, 480 mg of the crude product was dissolved in 27 mL of an acetone-water (8:1) solution. 207 mg of NMO and 47 μL of a 2.5% solution of OsO₄ in 2-methyl-2-propanol were added to the resultant solution under argon, followed by stirring at room temperature for 24 hours. To the resultant mixture was added 10 mL of a 10% aqueous Na₂SO₃ solution, followed by stirring for 30 minutes. An evaporation process was conducted. The resultant residue was subjected to an extraction treatment with 25 mL of EtOAc. The thus obtained organic layer was washed with brine (3×10 mL) and dried over MgSO₄. After a concentration process under reduced pressure, a purification treatment employing silica gel column chromatography (EtOAc-hexane (1:1) as a mobile phase) was performed such that 3-(4,2′-dimethoxy-5′-propyl-biphenyl-3-yl)-propane-1,2-diol (compound 26) as a clear oil was obtained (92% yield).

### Detected Properties of the Title Compound:

¹H NMR (400 MHz, CDCl₃, δ): 7.41 (dd, J=8.4, 2.2 Hz 1H), 7.35 (d, J=2.2 Hz, 1H), 7.09 (m, 2H), 6.92 (d, J=8.4 Hz, 1H), 6.88 (dd, J=6.8, 2.1 Hz, 1H), 3.97 (m, 1H), 3.87 (s, 3H), 3.78 (s, 3H), 3.65 (m, 1H), 3.53 (dd, J=11.4, 6.2 Hz, 1H), 2.87 (m, 2H), 2.56 (t, J=7.4 Hz, 2H), 2.51 (b, 1H), 2.25 (b, 1H), 1.64 (m, 2H), 0.95 (t, J=7.4 Hz, 3H). ¹³C NMR (100.6 MHz, CDCl₃, δ): 156.5, 154.5, 135.0, 132.6, 131.4, 130.8, 129.7, 129.0, 127.9, 125.4, 111.1, 110.2, 72.3, 66.1, 55.7, 55.6, 37.2, 34.6, 24.8, 13.9. IR (neat): 2957, 2930, 1805, 1508, 1492, 1463, 1268, 1244, 1166, 1134, 1065, 1027, 815 cm⁻¹. EIMS (70 eV) m/z: 330.2 (M⁺, 100), 269.2 (44.4), 241.1 (66.2), 239.1 (28.9), 224.1 (17.9), 195.1 (21.6), 165.1 (18.9), 152.1 (15.1). HRMS-EI (m/z): M⁺ calc. for C₂₀H₂₆O₄: 330.1831. found: 330.1833.

### Step II. Preparation of 4-(4,2′-dihydroxy-5′-propyl-biphenyl-3-ylmethyl)-[1,3]dioxolan-2-one (Compound 27)

402 mg of the 3-(4,2′-dimethoxy-5′-propyl-biphenyl-3-yl)-propane-1,2-diol (compound 26) as obtained in Step I of this example and 1.5 mL of pyridine were added into 20 mL of dichloromethane. 1.2 mL of a 20% solution of COCl₂ in toluene was added to the resultant solution at −78° C. under argon, followed by stirring at room temperature for 1 hour (TLC monitoring). 10 mL of dichloromethane was added in the resultant mixture. The resultant organic layer was washed with brine (3×10 mL) and dried over MgSO₄. A concentration process under reduced pressure was then conducted. Accordingly, a crude carbonate derivative was acquired (95% yield).

The crude carbonate derivative was added into 20 mL of dichloromethane. 5.6 mL of a 1 M solution of BBr₃ in dichloromethane was slowly added into the resultant solution under argon, followed by stirring at room temperature for 30 minutes. 5 mL of a saturated NaHCO₃ solution was added to the resultant mixture, followed by stirring for 5 minutes. The thus obtained organic layer was washed with brine (3×10 mL), dried over MgSO₄, and subjected to a concentration process. The resultant crude residue was subjected to a purification treatment employing silica gel column chromatography (EtOAc-hexane (3:7) as a mobile phase). Therefore, 4-(4,2′-Dihydroxy-5′-propyl-biphenyl-3-ylmethyl)-[1,3]dioxolan-2-one (compound 27) as an oil was obtained (91% yield).

### Detected Properties of the Title Compound:

¹H NMR (400 MHz, CDCl₃, δ): 7.27 (m, 2H), 7.01 (m, 1H), 6.99 (m, 1H), 6.87 (d, J=2.8 Hz, 1H), 6.85 (d, J=2.8 Hz, 1H), 5.77 (brs, 1H), 5.14 (brs, 1H), 5.07 (m, 1H), 4.47 (t, J=8.2 Hz, 1H), 4.33 (dd, J=8.6, 6.5 Hz, 1H), 3.22 (dd, J=14.0, 5.9 Hz, 1H), 3.06 (dd, J=14.0, 6.5 Hz, 1H), 2.53 (t, J=7.8 Hz, 2H), 1.62 (m, 2H), 0.94 (t, J=7.4 Hz, 3H). ¹³C NMR (100.6 MHz, CDCl₃, δ): 155.5, 153.5, 150.3, 135.2, 132.7, 130.4, 130.2, 129.7, 128.8, 127.0, 121.2, 116.1, 115.7, 76.4, 69.1, 37.2, 34.2, 24.8, 13.9. IR (neat): 3401, 2969, 1739, 1497, 1365, 1228, 1216, 1064 cm⁻¹. EIMS (70 eV) m/z: 328.2 (M⁺, 49.6), 299.1 (19.1), 284.2 (100), 266.2 (34.6), 255.1 (55.3), 237.1 (87.3), 211.1 (65.4), 165.1 (30.3), 115.1 (18.2). HRMS-EI (m/z): M⁺ calc. for C₁₉H₂₀O₅: 328.1311. found: 328.1305.

### Step III. Preparation of 4-(4,2% bis-methoxymethoxy-5′-propyl-biphenyl-3-ylmethyl)-[1,3]dioxolan-2-one (Compound 28)

A suspension of NaH (35 mg, 60% in oil) in THF (3 mL) was cooled to 0° C. under argon. 200 mg of the 4-(4,2′-Dihydroxy-5′-propyl-biphenyl-3-ylmethyl)-[1,3]dioxolan-2-one (compound 27) as obtained in Step II of this example was added into 2 mL of THF to form a solution. The solution was slowly added to the aforesaid suspension, followed by stirring at 0° C. for 30 minutes. 0.12 mL of methoxymethylbromide was added dropwise to the resultant mixture via a syringe, followed by stirring for 1 hour. The reaction was quenched through addition of 0.5 mL of water. A dilution process with 20 mL of EtOAc was conducted. The diluent was washed with brine (3×10 mL), dried, and subjected to a concentration process. The resultant crude residue was subjected to a purification treatment employing silica gel column chromatography (EtOAc-hexane (3:7) as a mobile phase). Therefore, 4-(4,2′-Bis-methoxymethoxy-5′-propyl-biphenyl-3-ylmethyl)-[1,3]dioxolan-2-one (compound 28) as a thick gum was obtained (90% yield).

### Detected Properties of the Title Compound:

¹H NMR (400 MHz, CDCl₃, δ): 7.41 (dd, J=8.5, 2.1 Hz, 1H), 7.37 (d, J=2.1 Hz, 1H), 7.16 (d, J=8.4 Hz, 1H), 7.10 (m, 3H), 5.25 (s, 2H), 5.09 (s, 2H), 5.03 (m, 1H), 4.45 (t, J=8.2 Hz, 1H), 4.27 (dd, J=8.5, 6.7 Hz, 1H), 3.51 (s, 3H), 3.41 (5, 3H), 3.28 (dd, J=13.6, 5.9 Hz, 1H), 3.04 (dd, J=13.6, 7.3 Hz, 1H), 2.56 (t, J=7.5 Hz, 2H), 1.62 (m, 2H), 0.95 (t, J=7.4 Hz, 3H). ¹³C NMR (100.6 MHz, CDCl₃, δ): 155.0, 154.3, 152.1, 136.6, 132.0, 130.7, 130.3, 130.0, 128.4, 122.2, 115.5, 113.6, 95.2, 94.5, 76.2, 68.8, 56.4, 56.1, 37.3, 34.6, 24.8, 13.9. IR (neat): 2957, 2930, 1807, 1489, 1236, 1197, 1156, 1127, 1077, 997, 922 cm⁻¹. EIMS (70 eV) m/z: 416.2 (M⁺, 100), 384.1 (18.6), 339.1 (42.9), 278.1 (62.9), 237.1 (28.3), 223.1 (26.4), 195.1 (18.0), 165.1 (20.4). HRMS-EI (m/z): M⁺ calc. for C₂₃H₂₈O₇: 416.1835. found: 416.1839.

### Step IV. Formation of Product 6 (Compound 29)

200 mg of the 4-(4,2′-Bis-methoxymethoxy-5′-propyl-biphenyl-3-ylmethyl)-[1,3]dioxolan-2-one (compound 28) as obtained in Step III of this example was dissolved in 15 mL of dioxane-H₂O (1:1). To the resultant solution was added a 20% solution of NaOH in water, followed by heating at 60° C. for 2 hours (TLC monitoring). The thus obtained mixture was cooled to room temperature, and the pH thereof was adjusted to 5-6 by adding dropwise 1 M HCl solution. The resultant turbid white residue was taken up in 15 mL of dichloromethane, followed by washing with brine (2×10 mL). A drying process and a concentration process were performed. Thus, a residue was formed (96% yield).

The residue was dissolved in 15 mL of MeOH, followed by cooling in an ice bath. A saturated aqueous solution of NaIO₄ (144 mg) was added dropwise to the resultant solution, thereby forming a turbid white residue. Stirring was conducted for 45 minutes (TLC monitoring). The resultant mixture was filtered over celite to remove all solids. An evaporation process was then performed. The thus acquired residue was subjected to an extraction treatment with 15 mL of EtOAc such that an extract was obtained. The extract was washed with brine and subjected to a concentration process under reduced pressure. A thick gummy residue was hence formed (85% yield).

The thick gummy residue was dried in vacuo. 108 mg of the vacuum dried residue was dissolved in 2 mL of dichloromethane. The resultant solution was slowly added to a cold orange suspension of CBr₄ (299 mg), PPh₃ (471 mg), and K₂CO₃ (248 mg) in dichloromethane (5 mL) at 10° C. under argon, followed by stirring for 15 minutes at 10° C. (TLC monitoring). The thus obtained mixture was diluted with 15 mL of dichloromethane, followed by performing a filtration treatment with a short pad of silica gel so as to remove triphenylphosphine oxide as much as possible. A concentration process was then conducted. Accordingly, yellow oil was formed (91% yield). The detected properties of the yellow oil were as follows: ¹H NMR (400 MHz, CDCl₃, δ): 7.35 (m, 2H), 7.10 (m, 4H), 6.61 (m, 1H), 5.25 (s, 2H), 5.10 (s, 2H), 3.51 (s, 3H), 3.48 (d, J=7.3 Hz, 2H), 3.43 (s, 3H), 2.56 (t, J=7.4 Hz, 2H), 1.62 (m, 2H), 0.95 (t, J=7.4 Hz, 3H). ¹³C NMR (100.6 MHz, CDCl₃, δ): 153.9, 152.1, 136.9, 136.5, 132.3, 131.2, 130.8, 130.7, 129.0, 128.8, 128.2, 125.8, 115.4, 113.4, 95.1, 94.3, 56.2, 37.3, 34.3, 24.7, 13.9.

117 mg of the aforesaid yellow oil was added into 5 mL of THF. 230 μL of a 16% solution of t-BuLi in pentane was added to the resultant solution at −78° C., followed by stirring at room temperature for 30 minutes (TLC monitoring). 0.5 mL of water was added. The resultant mixture was diluted with 10 mL of EtOAc. The diluent was washed with brine. A drying process and a concentration process were conducted, thereby forming a residue (99% yield). The residue was dissolved in 5 mL of THF, followed by addition of 0.5 mL of a concentrated HCl solution. The thus obtained mixture was stirred at room temperature for 36 hours (TLC monitoring). An evaporation process under reduced pressure was conducted, and an extraction treatment with 15 mL of EtOAc was preformed. The resultant organic layer was washed with brine (3×10 mL), dried over MgSO₄, and subjected to a concentration process. The crude product was subjected to a purification treatment employing silica gel column chromatography (EtOAc-hexane (1:5) as a mobile phase). Consequently, 5-Propyl-3′-prop-2-ynyl-biphenyl-2,4′-diol (product 6 or compound 29) as a gum was obtained (75% yield).

### Detected Properties of the Title Compound:

¹H NMR (400 MHz, CDCl₃, δ): 7.47 (s, 1H), 7.24 (m, 1H), 7.04 (m, 2H), 6.90 (d, J=4.6 Hz, 1H,), 6.88 (d, J=4.6 Hz, 1H), 5.74 (b, 1H), 5.14 (brs, 1H), 3.63 (d, J=2.3 Hz, 2H), 2.54 (t, J=7.5 Hz, 2H), 2.26 (s, 1H), 1.62 (m, 2H), 0.95 (t, J=7.4 Hz, 3H). ¹³C NMR (100.6 MHz, CDCl₃, δ): 153.3, 150.4, 135.0, 130.3, 130.2, 129.9, 129.0, 128.7, 127.4, 123.2, 116.4, 115.5, 80.9, 71.6, 37.2, 24.8, 19.9, 13.9. IR (neat): 3293, 2957, 2927, 2870, 2095, 1610, 1492, 1365, 1273, 1717, 1102, 822 cm⁻¹. EIMS (70 eV) m/z: 266.1 (M⁺, 100), 251.0 (57.3), 237.1 (76.5), 197.0 (13.8), 165.0 (18.6), 118.0 (23.9). HRMS-EI (m/z): M⁺ calc. for C₁₈H₁₈O₂: 266.1307. found: 266.1312.

### Synthesis Ex. 7 and Ex. 8

### 2,3′-Diallyl-biphenyl-3,4′-diol (Product 7 or Compound 34) and 4,3′-diallyl-biphenyl-3,4′-diol (Product 8 or Compound 35)

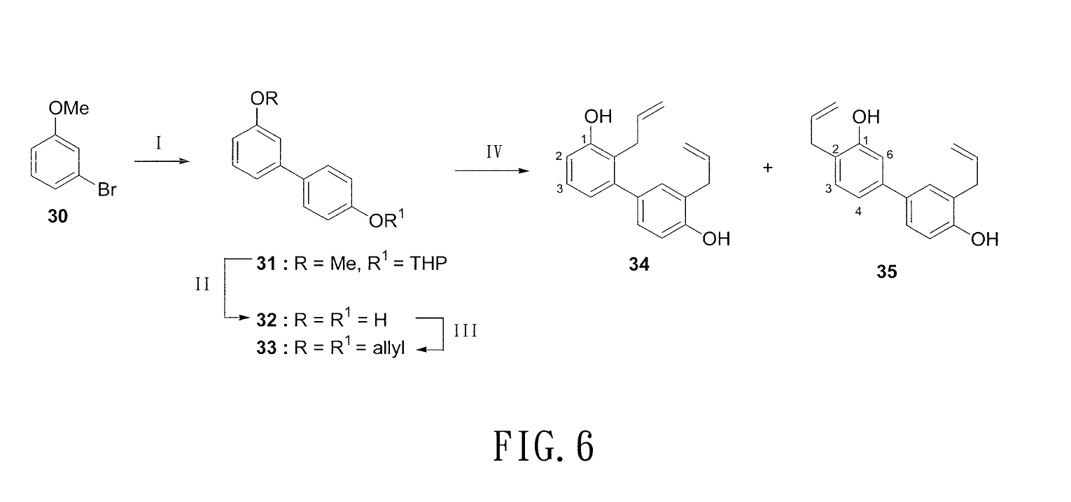

Syntheses of products 7 and 8 are outlined in FIG. 6.

### Step I. Preparation of 2-(3′-methoxy-biphenyl-4-yloxy)-tetrahydro-pyran (Compound 31)

400 mg of 3-bromo anisole (compound 30) (Aldrich) and 4-(tetrahydro-2H-pyran-2-yloxy)-phenylboronic acid pinacol ester (compound 4) (780 mg) were subjected to Suzuki-Miyaura reaction generally according to the method as described in Step II of Synthesis Example 1. The resultant crude product was subjected to a purification treatment employing silica gel column chromatography (EtOAc-hexane (1:9) as a mobile phase). Thus, 2-(3′-methoxy-biphenyl-4-yloxy)-tetrahydro-pyran (compound 31) as a gum was obtained (80% yield).

### Detected Properties of the Title Compound:

¹H NMR (400 MHz, CDCl₃, δ): 7.53 (m, 2H), 7.34 (t, J=7.9 Hz, 1H), 7.17-7.11 (m, 4H), 6.87 (dd, J=2.6 Hz, J=8.1, 1H), 5.49 (t, J=3.2 Hz, 1H), 3.97 (m, 1H), 3.87 (s, 3H), 3.65 (m, 1H), 2.1 (m, 1H), 1.90 (m, 2H), 1.72-1.68 (m, 3H). ¹³C NMR (100.6 MHz, CDCl₃, δ): 159.9, 156.8, 142.4, 134.5, 129.7, 128.2, 119.4, 116.7, 112.6, 112.1, 96.4, 62.1, 55.3, 30.4, 25.3, 18.8. IR (neat): 2943, 2873, 1608, 1571, 1515, 1480, 1453, 1438, 1356, 1295, 1268, 1212, 1177, 1123, 1110, 1050, 1036, 965, 920, 832 cm⁻¹. EIMS (70 eV) m/z: 284.2 (M⁺, 0.89), 214.1 (1.2), 200.1 (100), 186.1 (2.3), 171.1 (5.7), 157.1 (7.8), 139.0 (3.8), 128.1 (8.3), 115.0 (3.2), 85.0 (18.2), 67.0 (4.3), 55.0 (2.9). HRMS-EI (m/z): M⁺ calc. for C₁₈H₂₀O₃: 284.1412. found: 284.1405.

### Step II. Preparation of biphenyl-3,4′-diol (Compound 32)

The THP group of the 2-(3′-Methoxy-biphenyl-4-yloxy)-tetrahydro-pyran (compound 31) (350 mg) as obtained in Step I of this example was cleaved using PTSA generally according to the method described in Step II of Synthesis Example 2, followed by a demethylation reaction with BBr₃. Therefore, biphenyl-3,4′-diol (compound 32) as a solid was obtained (90% yield over two steps).

### Detected Properties of the Title Compound:

M.p.: 200° C. (decomposed). ¹H NMR (400 MHz, CD₃OD, δ): 7.45 (m, 2H), 7.23 (t, J=7.9 Hz, 1H), 7.03 (m, 2H), 6.87 (m, 2H), 6.76-6.74 (m, 1H). ¹³C NMR (100.6 MHz, CD₃OD, δ): 157.3, 156.7, 142.5, 132.5, 129.3, 127.6, 117.5, 115.1, 112.9, 112.8. IR (KBr): 3447, 1738, 1637, 1365, 1217, 749 cm⁻¹. EIMS (70 eV) m/z: 187.1 (M⁺+1, 100), 186.1 (85.4), 158.1 (19.5), 157.1 (18.2), 129.1 (12.8), 128.1 (15.6), 78.0 (7.8), 77.0 (11.4), 63.0 (13.0). HRMS-EI (m/z): M⁺ calc. for C₁₂H₁₀O₂: 186.0681. found: 186.0689.

### Step III. Preparation of 3,4′-bis-allyloxy-biphenyl (Compound 33)

200 mg of the biphenyl-3,4′-diol (compound 32) as obtained in Step II of this example was subjected to an allylation reaction generally according to Step IV of Synthesis Example 1. 3,4′-bis-allyloxy-biphenyl (compound 33) as a solid was hence obtained (87% yield).

### Detected Properties of the Title Compound:

M.p.: 120-122° C. ¹H NMR (400 MHz, CDCl₃, δ): 7.51 (m, 2H), 7.32 (t, J=7.8 Hz, 1H), 7.15-7.10 (m, 2H), 6.98 (m, 2H), 6.87 (dd, J=3.8, 8.2 Hz, 1H), 6.07 (m, 2H), 5.44 (m, 2H), 5.30 (m, 2H), 4.58 (m, 4H). ¹³C NMR (100.6 MHz, CDCl₃, δ): 158.9, 158.3, 142.3, 133.7, 133.3, 133.2, 129.7, 128.2, 119.4, 117.8, 117.7, 115.0, 113.4, 112.9, 68.9, 68.8. IR (neat): 3022, 2918, 1738, 1605, 1571, 1517, 1426, 1365, 1304, 1248, 1215, 1186, 1018, 993, 938, 832, 779 cm⁻¹. EIMS (70 eV) m/z: 266.2 (M⁺, 71.5), 225.1 (100), 197.1 (16.2), 169.1 (8.3), 156.1 (12.3), 139.1 (14.7), 128.1 (31.6). HRMS-EI (m/z): M⁺ calc. for C₁₈H₁₈O₂: 266.1307. found: 266.1315.

### Step IV. Formation of products 7 and 8 (Compounds 34 and 35)

200 mg of the 3,4′-bis-allyloxy-biphenyl (compound 33) as obtained in Step III of this example was dissolved in 10 mL of dichloromethane. To the resultant solution was added 1.88 mL of a 1 M solution of Et₂AlCl in hexane, followed by stirring for 3 hours (TLC monitoring). The reaction was carefully quenched by adding 1 N HCl at 0° C. and dichloromethane. The resultant organic layer was washed with brine (3×10 mL), dried over MgSO₄, and subjected to a concentration process. Accordingly, a crude product was formed. The crude product was subjected to a purification treatment employing silica gel column chromatography (EtOAc-hexane (1:9) as a mobile phase). Therefore, 2,3′-diallyl-biphenyl-3,4′-diol (product 7 or compound 34) as a solid and 4,3′-diallyl-biphenyl-3,4′-diol (product 8 or compound 35) as a gum were obtained (a combined yield of 95%).

### Detected Properties of the Title Compounds:

### Product 7

M.p.: 78-79° C. ¹H NMR (400 MHz, CDCl₃, δ): 7.16 (t, J=7.8 Hz, 1H), 7.07 (m, 2H), 6.85 (m, 3H), 6.04 (m, 2H), 5.19 (m, 5H), 5.09 (dd, J=15.5, 1.7 Hz, 1H), 3.44 (d, J=6.3 Hz, 2H), 3.37 (d, J=5.5 Hz, 2H). ¹³C NMR (100.6 MHz, CDCl₃, δ): 154.7, 153.3, 143.6, 136.8, 136.3, 134.2, 131.2, 128.4, 127.4, 125.0, 122.9, 122.8, 116.4, 116.2, 115.4, 114.8, 35.0, 32.1. IR (neat): 3435, 1578, 1508, 1460, 1203, 1115, 915, 786 cm⁻¹. EIMS (70 eV) m/z: 266.1 (M⁺, 100), 251.1 (34.6), 225.1 (60.3), 210.1 (80.2), 197.1 (38.0), 181.1 (40.1), 165.1 (37.4), 152.0 (32.3), 139.0 (16.5), 128.0 (13.5), 115.0 (29.8), 77.0 (19.8), 63.0 (10.6), 51.0 (11.6). HRMS-EI (m/z): M⁺ calc. for C₁₈H₁₈O₂: 266.1307. found: 266.1317.

### Product 8

¹H NMR (400 MHz, CDCl₃, δ): 7.32 (m, 2H), 7.15 (d, J=7.8 Hz, 1H), 7.08 (dd, J=7.8, 1.4 Hz, 1H), 7.01 (s, 1H), 6.86 (d, J=8.9 Hz, 5H), 6.05 (m, 2H), 5.27 (b, 2H), 5.20 (m, 4H). ¹³C NMR (100.6 MHz, CDCl₃, δ): 154.3, 153.7, 140.9, 136.5, 136.3, 133.5, 130.8, 129.1, 136.3, 123.8, 119.3, 116.7, 116.6, 116.5, 116.1, 114.1, 35.2, 34.9. IR (neat): 3429, 3078, 2922, 1637, 1610, 1576, 1494, 1461, 1534, 1405, 1236, 1116, 996, 916, 815 cm⁻¹. EIMS (70 eV) m/z: 266.1 (M⁺, 73), 241.2 (100), 199.2 (24.5), 181.2 (5.1), 165.1 (4.3), 106.1 (9.0). HRMS-EI (m/z): M⁺ calc. for C₁₈H₁₈O₂: 266.1307. found: 266.1317.

### Synthesis Ex. 9

### 3′-Allyl-2-propyl-biphenyl-4,4′-diol (Product 9 or Compound 42)

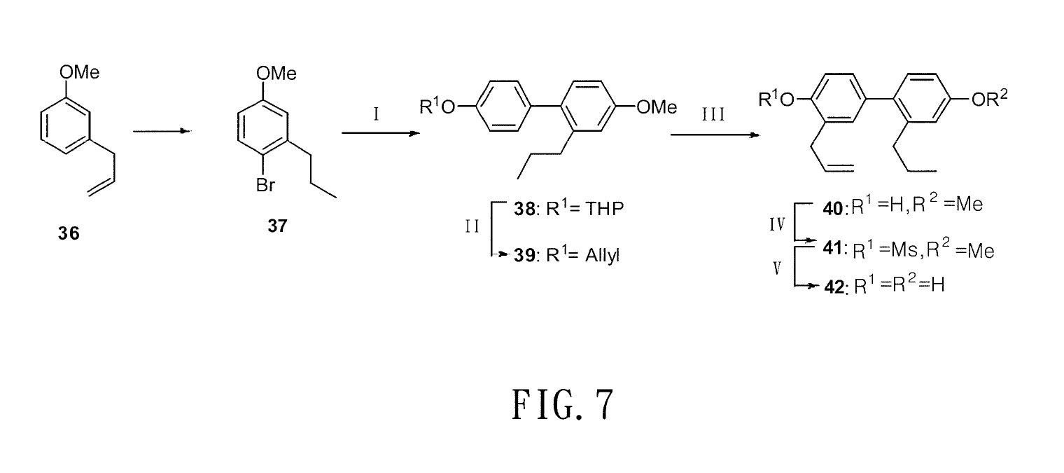

Synthesis of product 9 is outlined in FIG. 7.

### Step I. Preparation of 2-(4′-methoxy-2′-propyl-biphenyl-4-yloxy)-tetrahydro-pyran (Compound 38)

500 mg of 4-bromo-3-propyl anisole (compound 37), which can be prepared from 3-allyl anisole (compound 36) in two steps (i.e., sequential hydrogenation and bromination), and 4-(tetrahydro-2H-pyran-2-yloxy)-phenylboronic acid pinacol ester (compound 4) (795 mg) were subjected to Suzuki-Miyaura reaction generally according to the method as described in Step II of Synthesis Example 1, followed by a purification treatment employing silica gel column chromatography (EtOAc-hexane (1:9) as a mobile phase). Accordingly, 2-(4′-methoxy-2′-propyl-biphenyl-4-yloxy)-tetrahydro-pyran (compound 38) as an oil was obtained (89% yield).

### Detected Properties of the Title Compound:

¹H NMR (400 MHz, CDCl₃, δ): 7.19 (d, J=8.7 Hz, 2H), 7.12 (d, J=8.3 Hz, 1H), 7.08 (d, J=8.7 Hz, 2H), 6.83 (d, J=2.6 Hz, 1H), 6.77 (dd, J=8.3, 2.7 Hz, 1H), 5.46 (t, J=3.3 Hz, 1H), 3.99 (m, 1H), 3.84 (s, 3H), 3.69 (m, 1H), 2.54 (m, 2H), 2.05 (m, 1H), 1.89 (m, 2H), 1.66 (m, 3H), 1.51 (m, 2H), 0.84 (t, J=7.32 Hz, 3H). ¹³C NMR (100.6 MHz, CDCl₃, δ): 158.6, 155.9, 141.7, 135.1, 134.3, 131.2, 130.5, 115.9, 114.7, 110.7, 96.6, 62.3, 55.2, 35.4, 30.5, 25.3, 24.4, 18.9, 14.1. IR (neat): 2954, 2870, 1606, 1488, 1465, 1231, 1202, 1176, 1124, 1111, 1038, 966, 821 cm⁻¹. EIMS (70 eV) m/z: 326.2 (M⁺, 0.7), 242.1 (100), 213.1 (41.3), 182.1 (13.3), 85.0 (4.7). HRMS-EI (m/z): M⁺ calc. for C₂₁H₂₆O₃: 326.1882. found: 326.1888.

### Step II. Preparation of 4′-allyloxy-4-methoxy-2-propyl-biphenyl (Compound 39)

The THP group of the 2-(4′-methoxy-2′-propyl-biphenyl-4-yloxy)-tetrahydro-pyran (compound 38) (550 mg) as obtained in Step I of this example was cleaved using PTSA. The resultant phenol was subjected to an allylation reaction generally according to the method described in Step II of Synthesis Example 2. The thus acquired crude product was subjected to a purification treatment employing silica gel column chromatography (EtOAc-hexane (1:10) as a mobile phase). Thus, 4′-allyloxy-4-methoxy-2-propyl-biphenyl (compound 39) as an oil was obtained (94% yield over two steps).

### Detected Properties of the Title Compound:

¹H NMR (400 MHz, CDCl₃, δ): 7.19 (d, J=8.6 Hz, 2H), 7.12 (d, J=8.3 Hz, 1H), 7.08 (d, J=8.8 Hz, 2H), 6.83 (d, J=2.6 Hz, 1H), 6.77 (dd, J=8.3, 2.7 Hz, 1H), 6.10 (m, 1H), 5.46 (d, J=17.2 Hz, 1H), 5.32 (d, J=10.5 Hz, 1H), 4.58 (m, 2H), 3.84 (s, 3H), 2.54 (m, 2H), 1.51 (m, 2H), 0.83 (t, J=7.3 Hz, 3H). ¹³C NMR (100.6 MHz, CDCl₃, δ): 158.6, 157.3, 141.8, 134.4, 131.2, 133.4, 131.2, 130.5, 117.7, 114.7, 114.2, 110.7, 68.9, 55.3, 35.4, 24.4, 14.1. IR (neat): 2958, 2931, 2869, 1606, 1488, 1464, 1286, 1231, 1176, 1161, 1046, 998, 928, 835 cm⁻¹. EIMS (70 eV) m/z: 282.2 (M⁺, 50.9), 241.2 (100), 212.1 (11.1), 169.1 (15.2), 141.1 (22.5), 115.1 (21.2). HRMS-EI (m/z): M⁺ calc. for C₁₉H₂₂O₂: 282.1620. found: 282.1618.

### Step III. Preparation of 3-allyl-4′-methoxy-2′-propyl-biphenyl-4-ol (Compound 40)

400 mg of the 4′-allyloxy-4-methoxy-2-propyl-biphenyl (compound 39) as obtained in Step II of this example was subjected to a rearrangement reaction generally according to the method described in Step II of Synthesis Example 3. Therefore, 3-allyl-4′-methoxy-2′-propyl-biphenyl-4-ol (compound 40) as an oil was obtained (96% yield).

### Detected Properties of the Title Compound:

¹H NMR (400 MHz, CDCl₃, δ): 7.12 (d, J=3.0 Hz, 1H), 7.04 (m, 2H), 6.83 (m, 2H), 6.76 (dd, J=8.3, 2.7 Hz, 1H), 6.05 (m, 1H), 5.18 (m, 2H), 4.98 (b, 1H), 3.84 (s, 3H), 3.44 (d, J=6.2 Hz, 2H) 2.52 (m, 2H), 1.51 (m, 2H), 0.85 (t, J=7.3 Hz, 3H). ¹³C NMR (100.6 MHz, CDCl₃, δ): 158.6, 152.8, 141.8, 136.4, 134.5, 134.3, 131.6, 131.1, 128.8, 124.8, 116.5, 115.4, 114.7, 110.7, 55.3, 35.5, 35.1, 24.5, 14.1. IR (neat): 3420, 2958, 2930, 2869, 1606, 1488, 1465, 1231, 1162, 1049, 813 cm⁻¹. EIMS (70 eV) m/z: 282.2 (M⁺, 100), 241.2 (100), 253.2 (16.2), 212.1 (42.7), 197.1 (30.5), 165.1 (12.3), 152.1 (10.0), 115.1 (8.5). HRMS-EI (m/z): M⁺ calc. for C₁₉H₂₂O₂: 282.1620. found: 282.1613.

### Step IV. Preparation of Methanesulfonic Acid 3-allyl-4′-methoxy-2′-propyl-biphenyl-4-yl ester (Compound 41)

380 mg of the 3-allyl-4′-methoxy-2′-propyl-biphenyl-4-ol (compound 40) as obtained in Step III of this example was added into 15 mL of dichloromethane. To the resultant solution was added 0.5 mL of TEA. The thus obtained mixture was cooled in an ice bath for 30 minutes, followed by adding 150 μL of methanesulphonyl chloride. Stirring at room temperature was performed for 2 hours. The dichloromethane layer was washed with NaHCO₃ and brine (3×10 mL), dried over MgSO₄, and subjected to a concentration process under reduced pressure. The resultant crude residue was subjected to a purification treatment employing silica gel column chromatography (EtOAc-hexane (3:17) as a mobile phase). Accordingly, methanesulfonic acid 3-allyl-4′-methoxy-2′-propyl-biphenyl-4-yl ester (compound 41) as a gum was obtained (95% yield).

### Detected Properties of the Title Compound:

¹H NMR (400 MHz, CDCl₃, δ): 7.34 (d, J=8.3 Hz, 1H), 7.19 (b, 1H), 7.16 (dd, J=8.3, 2.0 Hz, 1H), 7.09 (d, J=8.3 Hz, 1H), 6.82 (d, J=2.5 Hz, 1H), 6.77 (dd, J=8.3, 2.5 Hz, 1H), 5.95 (m, 1H), 5.10 (m, 2H), 3.83 (s, 3H), 3.51 (d, J=6.6 Hz, 2H), 3.23 (s, 3H), 2.49 (m, 2H), 1.51 (m, 2H), 0.83 (t, J=7.3 Hz, 3H). ¹³C NMR (100.6 MHz, CDCl₃, δ): 159.1, 146.0, 141.6, 140.9, 135.6, 133.2, 132.4, 132.2, 130.9, 128.8, 121.6, 116.9, 114.8, 110.9, 55.3, 38.3, 35.3, 34.2, 24.5, 14.1. IR (neat): 2959, 1607, 1482, 1367, 1232, 1177, 1161, 969, 886, 839 cm⁻¹. EIMS (70 eV) m/z: 360.2 (M⁺, 48.5), 281.2 (100), 239.1 (14.6), 211.1 (14.2), 165.1 (12.1), 152.1 (7.7), 115.1 (5.5), 79.0 (10.6). HRMS-EI (m/z): M⁺ calc. for C₂₀H₂₄SO₄: 360.1395. found: 360.1398.

### Step V. Formation of Product 9 (Compound 42)

260 mg of the methanesulfonic acid 3-allyl-4′-methoxy-2′-propyl-biphenyl-4-yl ester (compound 41) as obtained in Step IV of this example was subjected to a demethylation reaction generally according to the method described in Step VI of Synthesis Example 1 such that a crude residue was formed. 220 mg of the crude residue was dissolved in 10 mL of dioxane-H₂O (1:1). 2 mL of a 20% aqueous NaOH solution was added into the resultant solution, followed by heating at 60° C. for 2 hours. The resultant mixture was neutralized via addition of 6 M HCl, thereby forming a turbid white residue. The turbid white residue was subjected to an extraction treatment with dichloromethane (3×10 mL), followed by washing with a saturated NaHCO₃ solution and brine. The resultant organic layer was dried over MgSO₄ and subjected to a concentration process. A purification treatment employing silica gel column chromatography (EtOAc-hexane (1:5) as a mobile phase) was conducted such that 3′-allyl-2-propyl-biphenyl-4,4′-diol (product 9 or compound 42) as a solid was obtained (80% yield over two steps).

### Detected Properties of the Title Compound:

M.p.: 76-78° C. ¹H NMR (400 MHz, CDCl₃, δ): 7.06 (d, J=8.3 Hz, 1H), 7.03 (m, 2H), 6.83 (d, J=8.7 Hz, 1H), 6.76 (d, J=2.5 Hz, 1H), 6.69 (dd, J=8.2, 2.6 Hz, 1H), 6.05 (m, 1H), 5.17 (m, 4H), 3.44 (d, J=6.4 Hz, 2H), 2.50 (m, 2H), 1.49 (m, 2H), 0.83 (t, J=7.3 Hz, 3H). ¹³C NMR (100.6 MHz, CDCl₃, δ): 154.5, 152.7, 142.0, 136.4, 134.5 134.4, 131.6, 131.3, 128.8, 124.9, 116.5, 115.8, 115.4, 112.5, 35.3, 35.0, 24.3, 14.0. IR (neat): 3367, 2958 2929, 2869, 1606, 1487, 148, 1232, 1165, 815 cm⁻¹. EIMS (70 eV) m/z: 268.1 (M⁺, 100), 257.1 (63.1), 198.1 (35.2), 165.1 (11.0), 115.0 (6.4), 83.1 (7.1). HRMS-EI (m/z): M⁺ calc. for C₁₈H₂₀O₂: 268.1463. found: 268.1459.

### Synthesis Ex. 10

### 2,3′-Dipropyl-biphenyl-4,4′-diol (Product 10 or Compound 53)

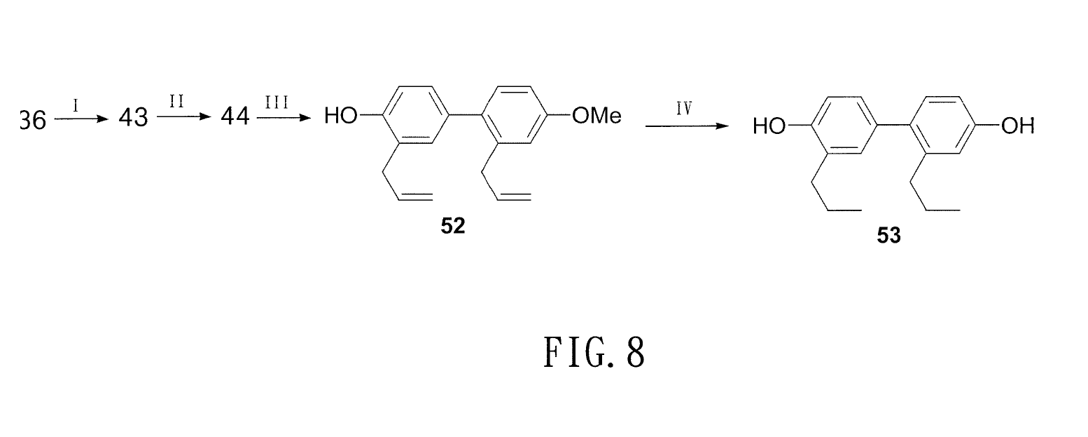

Synthesis of product 10 is outlined in FIG. 8.

### Step I. Preparation of 3-(3-methoxy-phenyl)-propane-1,2-diol (Compound 43)

1.5 g of 3-allyl anisole (compound 36) was subjected to a dihydroxylation reaction using OsO₄ generally according to the method as described in Step I of Synthesis Example 1 such that 3-(3-methoxy-phenyl)-propane-1,2-diol (compound 43) as a solid was obtained (91% yield).

### Detected Properties of the Title Compound:

M.p.: 90-92° C. ¹H NMR (400 MHz, CDCl₃, δ): 7.22 (t, J=7.7 Hz, 1H), 6.79 (m, 3H), 3.93 (m, 1H), 3.79 (s, 3H), 3.67 (m, 1H), 3.50 (m, 1H), 2.73 (m, 2H), 2.32 (m, 2H). ¹³C NMR (100.6 MHz, CDCl₃, δ): 159.8, 139.3, 129.7, 121.6, 115.6, 115.1, 111.9, 72.9, 66.1, 55.2, 39.9. IR (neat): 3391, 2939, 2836, 1602, 1585, 1489, 1455, 1437, 1259, 1153, 1092, 1038, 781, 697 cm⁻¹. EIMS (70 eV) m/z: 182.0 (M⁺, 27.6), 151.0 (21.2), 122.0 (100), 121.0 (44.8), 107.0 (24.7), 91.0 (11.8), 77.0 (20.7). HRMS-EI (m/z): M⁺ calc. for C₁₀H₁₄O₃: 182.0943. found: 182.0948.

### Step II. Preparation of 4-(2-bromo-5-methoxy-benzyl)-2,2-dimethyl-[1,3]dioxolane (Compound 44)

1.4 g of the 3-(3-methoxy-phenyl)-propane-1,2-diol (compound 43) as obtained in Step I of this example was added into 20 mL of dry acetone. PTSA (as a catalyst) was added to the resultant solution under argon, followed by stirring at room temperature for 5.5 hours (TLC monitoring). An evaporation process under reduced pressure was conducted, thereby forming a crude oily residue (95% yield).

The crude oily residue was dissolved in 5 mL of dry DMF. To the resultant solution was added 1.42 g of NBS, followed by stirring at room temperature for 18 hours. A filtration treatment was performed so as to remove solids. The resultant filtrate was poured into 50 mL of water. An extraction treatment with EtOAc-hexane (1:5) was then conducted. The resultant organic layer was washed with brine (3×10 mL), dried (MgSO₄), and subjected to a concentration process. Therefore, 4-(2-bromo-5-methoxy-benzyl)-2,2-dimethyl-[1,3]dioxolane (compound 44) as an oil was obtained (90% yield).

### Detected Properties of the Title Compound:

¹H NMR (400 MHz, CDCl₃, δ): 7.41 (d, J=8.7 Hz, 1H), 6.84 (d, J=3.0 Hz, 1H), 6.66 (dd, J=8.7, 3.0 Hz, 1H), 4.41 (m, 1H), 4.01 (dd, J=8.2, 5.9 Hz, 1H), 3.77 (s, 3H), 3.69 (dd, J=8.2, 6.6 Hz, 1H) 3.04 (dd, J=13.8, 6.6 Hz, 1H), 2.93 (dd, J=13.8, 6.2 Hz, 1H), 1.44 (s, 3H), 1.35 (s, 3H). ¹³C NMR (100.6 MHz, CDCl₃, δ): 158.8, 138.0, 133.3, 117.3, 115.1, 113.8, 109.2, 75.1, 68.7, 55.4, 40.2, 27.1, 25.7. IR (neat): 2985, 2935, 1594, 1572, 1474, 1416, 1369, 1285, 1161, 1062, 1014 cm⁻¹. EIMS (70 eV) m/z: 302.0 (M⁺+2, 9.9), 300.0 (M⁺, 8.9), 286.9 (8.4), 284.9 (8.6), 146.0 (26.9), 101.0 (100), 73.1 (18.5). HRMS-EI (m/z): M⁺ calc. for C₁₃H₁₇BrO₃: 300.0361. found: 300.0365.

### Step III. Preparation of 3,2′-diallyl-4′-methoxy-biphenyl-4-ol (Compound 52)

1.5 g of the 4-(2-bromo-5-methoxy-benzyl)-2,2-dimethyl-[1,3]dioxolane (compound 44) as obtained in Step II of this example was added into 10 mL of MeOH. To the resultant solution was added 1 mL of 1 M HCl, followed by stirring at room temperature for 5 hours (TLC monitoring). An evaporation process was conducted. The resultant residue was subjected to an extraction treatment with 15 mL of EtOAc. The thus obtained extract was washed with brine (3×10 mL), dried over MgSO₄, and subjected to a concentration process. A crude diol was hence formed (100% yield).

500 mg of the crude diol and 4-(tetrahydro-2H-pyran-2-yloxy)-phenylboronic acid pinacol ester (compound 4) (697 mg) were subjected to Suzuki-Miyaura reaction generally according to the method as described in Step II of Synthesis Example 1, followed by a purification treatment employing silica gel column chromatography (EtOAc-hexane (3:2) as a mobile phase). A coupling product was hence obtained (76% yield). 323 mg of the coupling product was converted to the corresponding phenol derivative (92% yield) generally according to the method as described in Step III of Synthesis Example 1. 164 mg of the corresponding phenol derivative was subjected to an allyllation reaction generally according to the method as described in Step IV of Synthesis Example 1, thereby forming an O-allylated derivative (94% yield). The O-allylated derivative was subjected to Claisen rearrangement using Et₂AlCl such that 3,2′-diallyl-4′-methoxy-biphenyl-4-ol (compound 52) as an oil was obtained (95% yield).

### Detected Properties of the Title Compound:

¹H NMR (400 MHz, CDCl₃, δ): 7.15 (d, J=8.2 Hz, 1H), 7.05 (m, 2H), 6.81 (m, 3H), 6.01 (m, 1H), 5.88 (m, 1H), 5.18 (m, 2H), 4.99 (m, 3H), 3.83 (s, 3H), 3.43 (d, J=6.3 Hz, 2H), 3.31 (d, J=6.3 Hz, 2H). ¹³C NMR (100.6 MHz, CDCl₃, δ): 158.7, 152.9, 138.7, 137.7, 136.4, 134.3, 134.1, 131.5, 131.2, 128.8, 124.8, 116.6, 115.9, 115.4, 114.9, 111.5, 55.3, 37.8, 35.1. IR (neat): 3429, 3002, 2933, 2835, 1606, 1488, 1431, 1231, 1161, 1051, 913, 813 cm⁻¹. EIMS (70 eV) m/z: 280.2 (M⁺, 100), 265.2 (26.7), 239.1 (24.2), 224.1 (69.5), 181.1 (18.4), 165.1 (18.5), 152.1 (15.3), 115.1 (11.3). HRMS-EI (m/z): M⁺ calc. for C₁₉H₂₀O₂: 280.1463. found: 280.1469.

### Step IV. Formation of Product 10 (Compound 53)

145 mg of the 3,2′-diallyl-4′-methoxy-biphenyl-4-ol (compound 52) as obtained in Step III of this example was subjected to a hydrogenation reaction generally according to the method described in Step V of Synthesis Example 2, followed by performing a demethylation reaction with BBr₃ generally according to the method described in Step VI of Synthesis Example 1. The resultant crude product was subjected to a purification treatment employing silica gel column chromatography (EtOAc-hexane (3:17) as a mobile phase). Thus, 2,3′-dipropyl-biphenyl-4,4′-diol (product 10 or compound 53) as an oil was obtained (92% yield over two steps).

### Detected Properties of the Title Compound:

¹H NMR (400 MHz, CDCl₃, δ): 7.04 (m, 2H), 7.05 (m, 2H), 6.96 (dd, J=8.2, 2.0 Hz, 1H), 6.79 (m, 2H), 6.71 (dd, J=8.2, 2.5 Hz, 1H), 2.63 (t, J=7.6 Hz, 2H), 2.50 (t, J=7.6 Hz, 2H), 1.67 (m, 2H), 1.49 (m, 2H), 0.98 (t, J=7.3 Hz, 3H), 0.79 (t, J=7.3 Hz, 3H). ¹³C NMR (100.6 MHz, CDCl₃, δ): 154.6, 152.3, 141.9, 134.4, 134.1, 131.5, 131.3, 127.9, 115.8, 114.8, 112.6, 35.3, 32.1, 24.4, 22.9, 14.1, 14.0. IR (neat): 3429, 3002, 2933, 2835, 1606, 1488, 1431, 1231, 1161, 1051, 913, 813 cm⁻¹. EIMS (70 eV) m/z: 270.1 (M⁺, 100), 241.1 (12.9), 199.0 (25.8), 181.1 (9.0). HRMS-EI (m/z): M⁺ calc. for C₁₈H₂₂O₂: 270.1620. found: 270.1613.

### Results:

The anisole compounds of formula (II) in the above examples, i.e., 3-(3-bromo-4-methoxy-phenyl)-propane-1,2-diol (compound 3), 2-bromo-4-propyl anisole, 3-bromo anisole (compound 30), 4-bromo-5-propyl anisole (compound 37), and 3-(2-bromo-5-methoxy-phenyl)-propane-1,2-diol, were prepared with good yield. The arylboronic compound of formula (III) in the above examples, i.e., 4-(tetrahydro-2H-pyran-2-yloxy)-phenylboronic acid pinacol ester (compound 4), can be easily prepared from the known boronic acid with excellent yield (Cladingboel D. E. (2000), Org. Process Res. Dev., 4: 153). The biphenyl compounds of formula (I′) in the above examples, i.e., 3-[6-methoxy-4′-(tetrahydro-pyran-2-yloxy)-biphenyl-3-yl]-propane-1,2-diol (compound 5), 2-(2′-methoxy-5′-propyl-biphenyl-4-yloxy)-tetrahydro-pyran (compound 10), 2-(3′-methoxy-biphenyl-4-yloxy)-tetrahydro-pyran (compound 31), 2-(4′-methoxy-2′-propyl-biphenyl-4-yloxy)-tetrahydro-pyran (compound 38), and 2-[4-methoxy-4′-(tetrahydro-pyran-2-yloxy)-biphenyl-3-yl]-propane-1,2-diol, were prepared with good yield using the coupling step of the method according to this invention (i.e., the Suzuki reaction employing the anisole compound without an allyl substituent and the arylboronic compound without an allyl substituent), which can prevent isomerization.

Honokiol (product 1 or compound 1) was synthesized with an overall yield of 45% using the method of this invention. The overall yield of honokiol resulting from the method of this invention is much higher than that resulting from the method of R. M. Denton et al. (21%). Therefore, the method according to the present invention is suitable for large-scale industrial production.

### PHARMACOLOGICAL EXAMPLES

In order to determine the biological activities of the biphenolic compounds of formula (I) in the treatment of Parkinson's disease, the following analyses were performed.

### Experimental Materials:

1. SH-SY5Y human neuroblastoma cells (American Type Culture Collection (ATCC), CRL-2266) were cultivated using a mixture of Dulbecco's Modified Eagle's Medium (DMEM)/F12 medium, which was supplemented with 10% fetal bovine serum, penicillin (100 U/mL), and streptomycin (100 μg/mL) and which had a pH of 7.4, and were incubated in a humidified atmosphere (95% air/5% CO₂, 37° C.). The SH-SY5Y neuroblastoma cells were seeded in a well-plate at a density of about 2×10⁵ cells/well for the analysis below.
2. Male NMRI mice (10-11 weeks old, 35-45 g) were obtained from the Laboratory Animal Center of Tzu Chi University (Hualien, Taiwan), were housed in cages (4-5 mice per cage), were maintained on a 12 hour light/dark cycle at 25±2° C., and were provided with food and water ad libitum. The experimental protocol was approved by Review Committee of the Tzu Chi University for the use of the animals.

### General Procedures:

### 1. MTT Assay:

3-(4,5-Dimethylthiazol-2-yl)-2,5-diphenyltetrazolium bromide (MTT) assay was conducted generally as follows. A MTT solution was added to culture of the cells in the respective well so that a MTT concentration of 0.5 mg/mL was reached, followed by cultivation for 4 hours. The liquid in the respective well was removed, and dimethylsulfoxide (DMSO) was added into the respective well to dissolve formazan dye. Absorbance of the mixture in the respective well at 570 nm (OD₅₇₀) was measured using an OPTImax microplate reader (Molecular Devices).

### 2. Preparation of Mouse Model of Parkinson's Disease (PD):

The respective NMRI mouse was anesthetized with ketamine (1 mg/kg, intraperitoneally). Subsequently, the respective NMRI mouse was placed into a stereotactic frame with nose and ear bars specially designed for mice. 6-hydroxydopamine (6-OHDA) was injected with saline containing 0.02% ascorbic acid (as a vehicle) into the right striatum of the respective NMRI mouse so that unilateral striatal lesion was induced (i.e., damage to dopaminergic neurons in the unilateral striatum was induced). The injection was conducted at a rate of 1 μL/minute using a Hamilton syringe at the following coordinates: AP: −0.9 mm; ML: −1.9 mm; DV: −2.2 mm with respect to bregma, and a final dose of 15 μg was administered. The needle was left standing for 5 minutes after the injection was finished, and was slowly withdrawn.

One week after the injection of 6-OHDA, an apomorphine-induced rotation test was performed for the respective NMRI mouse so as to confirm that damage to striatal dopaminergic neurons was successfully induced by 6-OHDA. The NMRI mouse exhibiting significantly more contralateral movements than ipsilateral movements was considered to be appropriately lesioned and served as a mouse model of PD.

### 3. Apomorphine-Induced Rotation Test:

The respective NMRI mouse was allowed to habituate to the ambient environment for 10 minutes before the apomorphine-induced rotation test was performed. Apomorphine was then injected intraperitoneally into the respective NMRI mouse at a dose of 0.5 mg/kg. The respective NMRI mouse was placed in a glass bowl with a diameter of 20 cm and was attached to an automatic rotometer (TSE, Bad Homburg, Germany) via a specially designed harness. Each circular movement exceeding 30° was recorded. The contralateral movements (toward the nonlesioned side) and ipsilateral movements (toward the lesioned side) of the NMRI mice were counted during a period of 60 minutes. The net rotation was calculated according to the following formula:

A=B−C (1)

where A=net rotation
B=the total number of ipsilateral movements during the period of 60 minutes
C=the total number of contralateral movements during the period of 60 minutes
   4. Determination of TH (Tyrosine Hydroxylase) Expression Level and iNOS (Inducible Nitric Oxide Synthase) Expression Level:

The respective NMRI mouse was deeply anaesthetized with thiopental (50 mg/kg, intraperitoneally). The respective NMRI mouse was perfused transcardially with a heparin solution (0.05% heparin in 0.1 M PBS), and was subsequently perfused with 100 mL of ice-cold fixative (4% paraformaldehyde in 0.1 M PBS). The brain of the respective NMRI mouse was removed and was postfixed in the same ice-cold fixative for 12 hours, followed by dehydration in 30% sucrose in 0.1 M PBS. The brain was frozen and was subjected to tissue slicing. Serial coronal sections (a thickness of 20 μm) were cut on a freezing microtome and were placed in PBS. The sections were later processed for free-floating TH or iNOS immunohistochemistry.

The sections were rinsed three times with PBS (30 minutes for each time), were treated with 3% H₂O₂/10% methanol in PBS for 30 minutes for quenching the endogenous peroxidase activity, and were rinsed three times with PBS. After preincubation with 0.5% normal goat serum (NGS)/0.2% Triton in PBS, the sections were incubated with a primary antibody (anti-TH antibody or anti-iNOS antibody, 1:5000, Novus Biologicals, Inc., Littleton, Colo., USA) in 0.5% NGS/PBS at 4° C. for 16-18 hours. After being rinsed three times with PBS, the sections were incubated with a biotinylated goat anti-rabbit IgG (1:200) in PBS for 1 hour. Subsequently, the sections were rinsed three times with PBS and were stained with 0.05% diaminobenzidine/0.03% H₂O₂ in PBS for 5 minutes. The respective section was placed on a gelatin-coated slide, dehydrated through ascending concentrations of alcohol, and covered with a slipcover for observation under a light microscope.

### 5. Statistical Analysis:

Data are expressed as mean±SEM. Data were subjected one-way analysis of variance (ANOVA) alone, or ANOVA and a Student-Newman-Keuls post-hoc test. Statistical significance is indicated by p<0.05.

### Pharmacological Ex. 1

### In Vitro Analysis for Neuroprotective Effects of Biphenolic Compounds of Formula (I)

In order to investigate whether the biphenolic compounds of formula (I) have potential therapeutic effects on Parkinson's disease, the following in vitro analyses were conducted. Products 1, 2, 3, 4, 5, 7 and 3,3′-diallyl-biphenyl-4,4′-diol (compound 55) were subjected to the following in vitro analyses.

### A. Analysis for Effects of Biphenolic Compounds of Formula (I) on Oxidative Stress

The SH-SY5Y human neuroblastoma cells were respectively treated with products 1, 2, 3, 4, 5, 7, and 3,3′-diallyl-biphenyl-4,4′-diol (compound 55) (10 μM) for 30 minutes. 3,3′-diallyl-biphenyl-4,4′-diol (compound 55) was prepared from commercially available 4,4′-biphenol (compound 54) generally according to the method as described in Ablard, F. et al., Bioorg. Med. Chem. Lett. 2007, 17, 4428. Subsequently, the SH-SY5Y human neuroblastoma cells were exposed to cumene hydroperoxide (CHP) or tert-butyl hydroperoxide (TBHP) (300 μM, Riedel-de Haen) for 3 hours after the treatment with the respective biphenolic compound of formula (I). SH-SY5Y human neuroblastoma cells that were not exposed to CHP or TBHP, and that did not receive the treatments with the biphenolic compounds of formula (I) served as a normal control. SH-SY5Y human neuroblastoma cells that were exposed to CHP or TBHP for 3 hours, and that did not receive the treatments with the biphenolic compounds of formula (I) served as a pathological control.

Cell viability was evaluated as follows. The SH-SY5Y human neuroblastoma cells were subjected to the MTT assay as described in section 1 of General Procedures. The relative cell viability (%) was calculated by substituting the absorbance into the following formula:

D=(E/F)×100 (2)

where D=relative cell viability
E=OD₅₇₀ of the pathological control, or the SH-SY5Y human neuroblastoma cells treated with the respective biphenolic compound of formula (I)
F=OD₅₇₀ of the normal control
   The obtained data were subjected to the statistical analysis as described in section 5 of General Procedures.

### B. Analysis for Effects of Biphenolic Compounds of Formula (I) on Neurotoxicity

SH-SY5Y human neuroblastoma cells were exposed to neurotoxic 1-methyl-4-phenylpyridinium (MPP⁺) (1 mM) for 24 hours, while the same were treated with the respective one of products 1, 2, 3, 4, 5, 7, and 3,3′-diallyl-biphenyl-4,4′-diol (compound 55) (10 μM or 30 μM). SH-SY5Y human neuroblastoma cells that were not exposed to MPP⁺, and that did not receive the treatments with the biphenolic compounds of formula (I) served as a normal control. SH-SY5Y human neuroblastoma cells that were exposed to MPP⁺ for 24 hours, and that did not receive the treatments with the biphenolic compounds of formula (I) served as a pathological control.

Cell viability was evaluated as follows. The SH-SY5Y human neuroblastoma cells were subjected to the MTT assay as described in section 1 of General Procedures. The relative cell viability (%) was calculated by substituting the absorbance into the following formula:

G=(H/I)×100 (3)

where G=relative cell viability
H=OD₅₇₀ of the pathological control, or the SH-SY5Y human neuroblastoma cells treated with the respective biphenolic compound of formula (I)
I=OD₅₇₀ of the normal control
   The obtained data were subjected to the statistical analysis as described in section 5 of General Procedures.

### Results:

### A. Analysis for Effects of Biphenolic Compounds of Formula (I) on Oxidative Stress

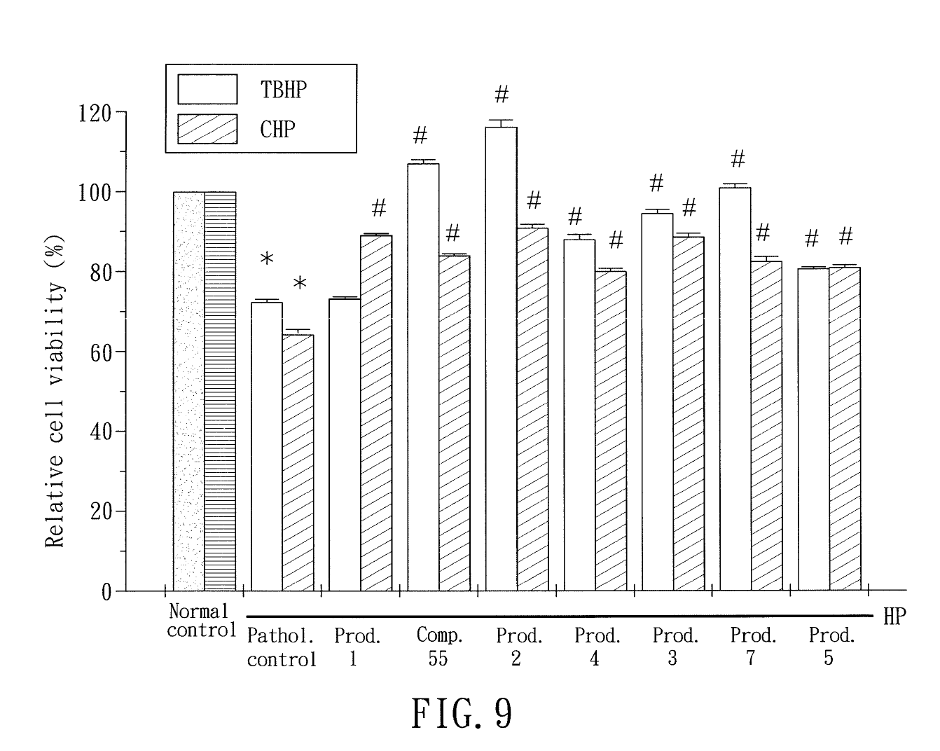

Referring to FIG. 9, the relative cell viability of the SH-SY5Y neuroblastoma cells of the pathological controls was 72% (exposure to TBHP) and 64% (exposure to CHP), respectively. Regarding the SH-SY5Y neuroblastoma cells exposed to TBHP, six of the seven biphenolic compounds, i.e., products 2, 3, 4, 5, 7, and 3,3′-diallyl-biphenyl-4,4′-diol (compound 55) significantly prevented cell death, and product 2 was the most effective in preventing cell death among them. Regarding the SH-SY5Y neuroblastoma cells exposed to CHP, all of the biphenolic compounds, i.e., products 1, 2, 3, 4, 5, 7, and 3,3′-diallyl-biphenyl-4,4′-diol (compound 55) significantly prevented cell death, and product 2 was the most effective in preventing cell death among them. The aforesaid experimental results indicate that the biphenolic compounds of formula (I) have different potencies and efficacies to prevent neuronal cell death caused by oxidative stress.

### B. Analysis for Effects of Biphenolic Compounds of Formula (I) on Neurotoxicity

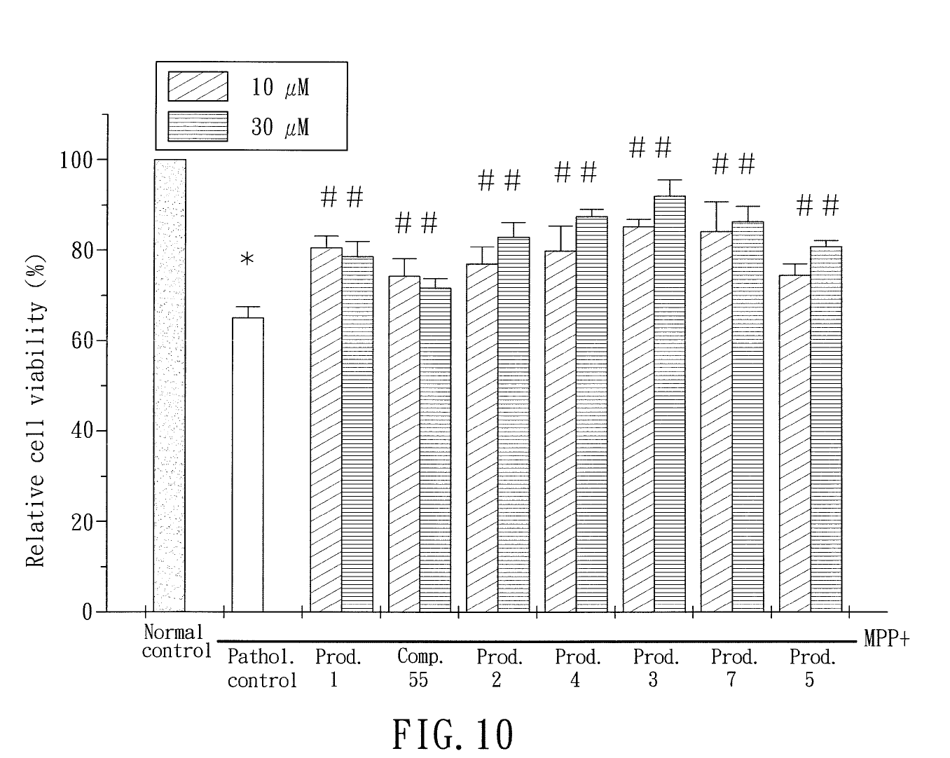

Referring to FIG. 10, the relative cell viability of the SH-SY5Y neuroblastoma cells of the pathological control was significantly lower than that of the SH-SY5Y neuroblastoma cells treated with the respective biphenolic compound of formula (I) (10 μM and 30 μM). Furthermore, products 2, 3, 4, 5, 7 alleviated the cytotoxicity of MPP⁺ in a concentration-dependent manner. The aforesaid experimental results reveal that the biphenolic compounds of formula (I) have a neuroprotective activity against MPP⁺-induced neuronal cell death.

### Pharmacological Ex. 2

### In Vivo Analysis for Therapeutic Effect of Biphenolic Compounds of Formula (I) on Parkinson's Disease

In order to examine whether the biphenolic compounds of formula (I) are effective in treating Parkinson's disease, the following in vivo experiments employing mouse models of PD were performed. Product 1 alone, or product 1 or 2 were subjected to the following in vivo experiments.

A. Effect of Subchronic Treatment with Biphenolic Compound of Formula (I) on Rotational Behavior and Reduction in TH Expression Induced by 6-OHDA

Product 1 was administered intraperitoneally to NMRI mice at a dose of 5 mg/kg or 10 mg/kg 30 minutes before 6-OHDA injection was preformed according to the method as described in section 2 of General Procedures. After the 6-OHDA injection, product 1 was administered intraperitoneally to some of the aforesaid NMRI mice daily at a dose of 5 mg/kg or 10 mg/kg for 14 consecutive days. In addition, the remaining NMRI mice were not treated with product 1 after the 6-OHDA injection.

NMRI mice into which a vehicle (saline containing 0.02% ascorbic acid) was injected (generally according to the method as described in section 2 of General Procedures, except that 6-OHDA was not injected), and which were not treated with product 1 before and after the injection of the vehicle, served as a sham operated control. NMRI mice that were subjected to 6-OHDA injection and that were not treated with product 1 before and after the 6-OHDA injection served as a pathological control.

One week after the injection of the vehicle or 6-OHDA, the NMRI mice were subjected to the apomorphine-induced rotation test as described in section 3 of General Procedures.

14 days after the injection of the vehicle or 6-OHDA, the NMRI mice were subjected to the apomorphine-induced rotation test as described in section 3 of General Procedures.

The obtained data were subjected to the statistical analysis as described in section 5 of General Procedures. TH expression level in the striatum was determined according to the method as described in section 4 of General Procedures.

### Results:

One week after the injection of 6-OHDA, the NMRI mice subjected to the injection of 6-OHDA into the right striatum exhibited significantly more contralateral movements than ipsilateral movements during the period of 60 minutes, i.e., an apomorphine-induced rotational behavior (data not shown). The NMRI mice of the sham operated control did not exhibit excessively more contralateral movements than ipsilateral movements one week after the injection of the vehicle (data not shown). Single administration of product 1 (5 mg/kg or 10 mg/kg) 30 minutes before the injection of 6-OHDA did not prevent the apomorphine-induced rotation behavior one week after the injection of 6-OHDA (data not shown).

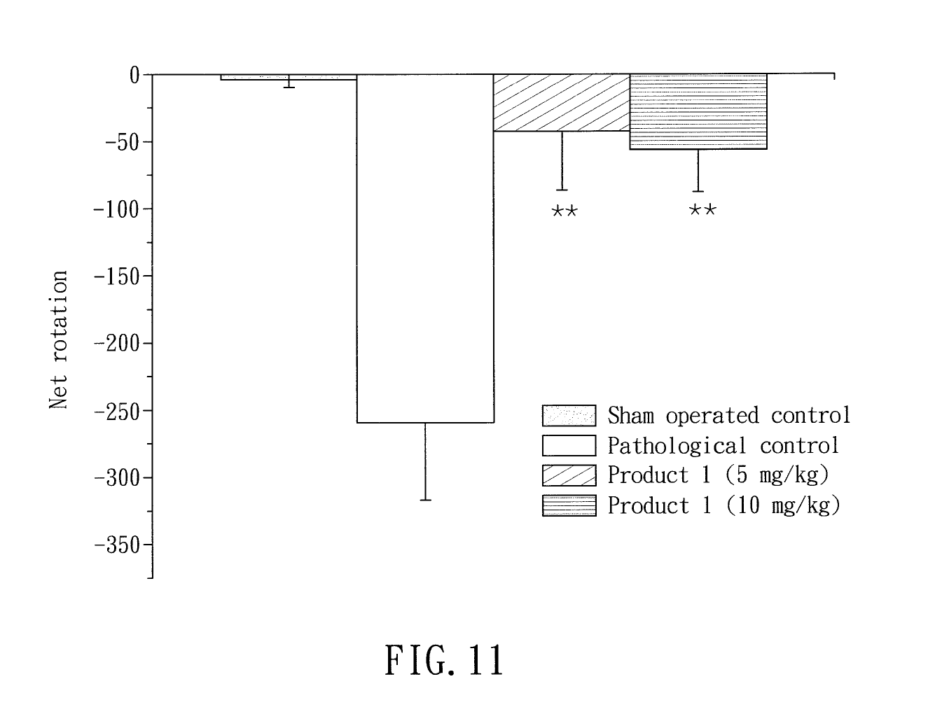

As shown in FIG. 11, the administration of honokiol (5 mg/kg or 10 mg/kg) for 14 consecutive days after the 6-OHDA injection significantly reduced the contralateral movements induced by apomorphine.

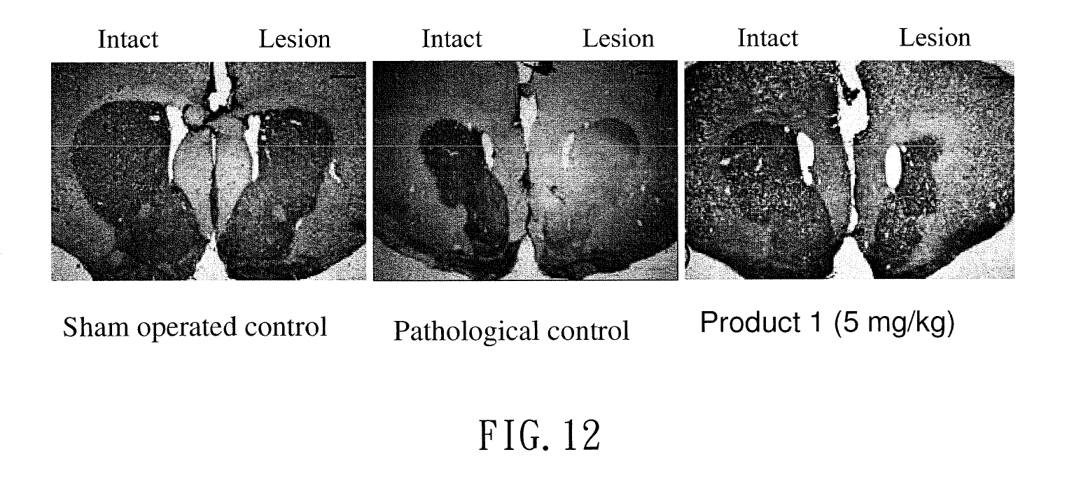

As shown in FIG. 12, dense tyrosine hydroxylase immunoreactivity (TH-ir) was observed in both of the striata of the NMRI mice of the sham operated control, but severe loss of TH-ir fibers was observed on the lesion site of the NMRI mice of the pathological control. The right striatum of the NMRI mice treated with product 1 (5 mg/kg) for 14 consecutive days after the 6-OHDA injection have a significantly higher TH expression level compared to that of the NMRI mice of the pathological control.

The aforesaid experimental results indicate the subchronic treatment with the biphenolic compounds of formula (I) can prevent the rotational behavior and reduction in TH expression induced by 6-OHDA.

B. Effects of Subchronic Post-Treatments with Biphenolic Compounds of Formula (I) on Rotational Behavior, Reduction in TH Expression, and iNOS Expression Induced by 6-OHDA

6-OHDA was injected into NMRI mice according to the method as described in section 2 of General procedures. Product 1 or product 2 was administered intraperitoneally to the respective NMRI mouse at a dose of 5 mg/kg, 1 mg/kg, 0.5 mg/kg, 0.1 mg/kg, or 0.05 mg/kg for 14 consecutive days, starting on Day 7 after the 6-OHDA injection. NMRI mice into which a vehicle (saline containing 0.02% ascorbic acid) was injected (generally according to the method as described in section 2 of General Procedures, except that 6-OHDA was not injected), and which were not treated with product 1 or product 2 after the injection of the vehicle, served as a sham operated control. NMRI mice into which 6-OHDA was injected and which were not treated with product 1 or product 2 after the 6-OHDA injection served as a pathological control. The rotational behavior of the NMRI mice was evaluated using the apomorphine-induced rotation test as described in section 3 of General Procedures on Days 7, 14, and 21 after the 6-OHDA injection.

TH expression level in the striatum and the substantia nigra (SN), and iNOS expression level in the striatum were determined according to the method as described in section 4 of General Procedures on Day 21 after the injection of 6-OHDA or the vehicle.

In addition, the optical density regarding the lesioned and unlesioned sites in the striatum and SN of the NMRI mice (e.g., the sham operated control, the pathological control, and the NMRI mice treated with product 1) was determined from the obtained photomicrographs illustrating TH expression level via ImageJ software (National Institutes of Health). The relative optical density was calculated using the following formula:

J=K/L (4)

where J=relative optical density
K=optical density of the lesioned site
L=optical density of the unlesioned site

Furthermore, photomicrographs illustrating iNOS expression level were subjected to cell count using ImageJ software (National Institutes of Health) so as to count cells with iNOS expression.

The obtained data were subjected to the statistical analysis as described in section 5 of General Procedures.

### Results:

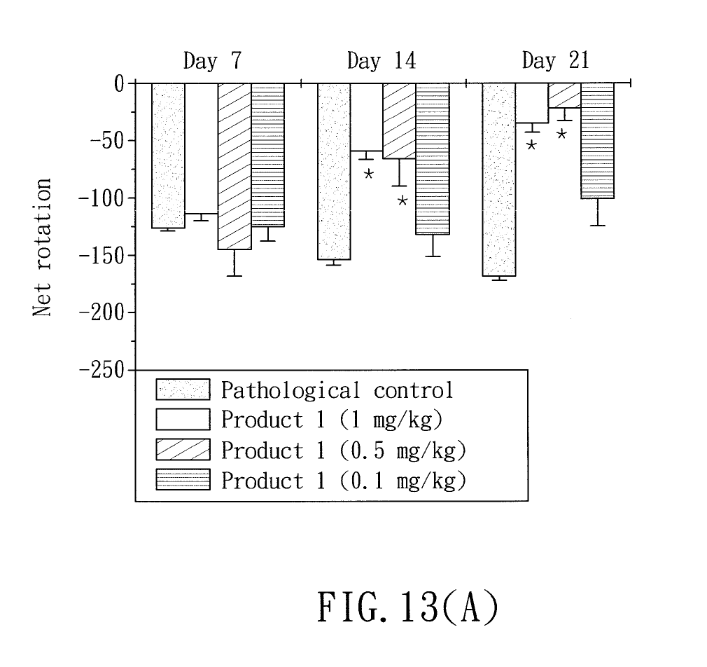

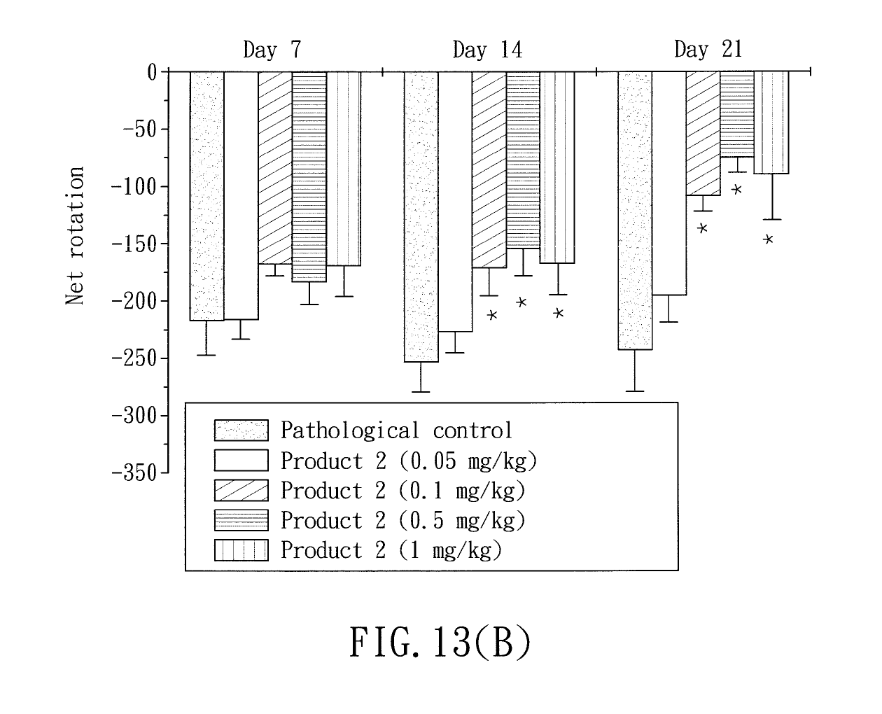

As shown in FIG. 13(A), 0.5 mg/kg and 1 mg/kg of product 1 significantly inhibited unilateral rotations. As shown in FIG. 13(B), 0.1 mg/kg, 0.5 mg/kg, and 1 mg/kg of product 2 significantly inhibited unilateral rotations. Therefore, a low dose of product 2 (i.e., 0.1 mg/kg), not a low dose of product 1 (i.e., 0.1 mg/kg), can still alleviate the apomorphine-induced rotational behavior. The aforesaid experimental results indicate that product 2 exhibits more potent restoring activity against neurotoxin-induced motor dysfunction than that of product 1 in the treatment of PD-like symptoms.

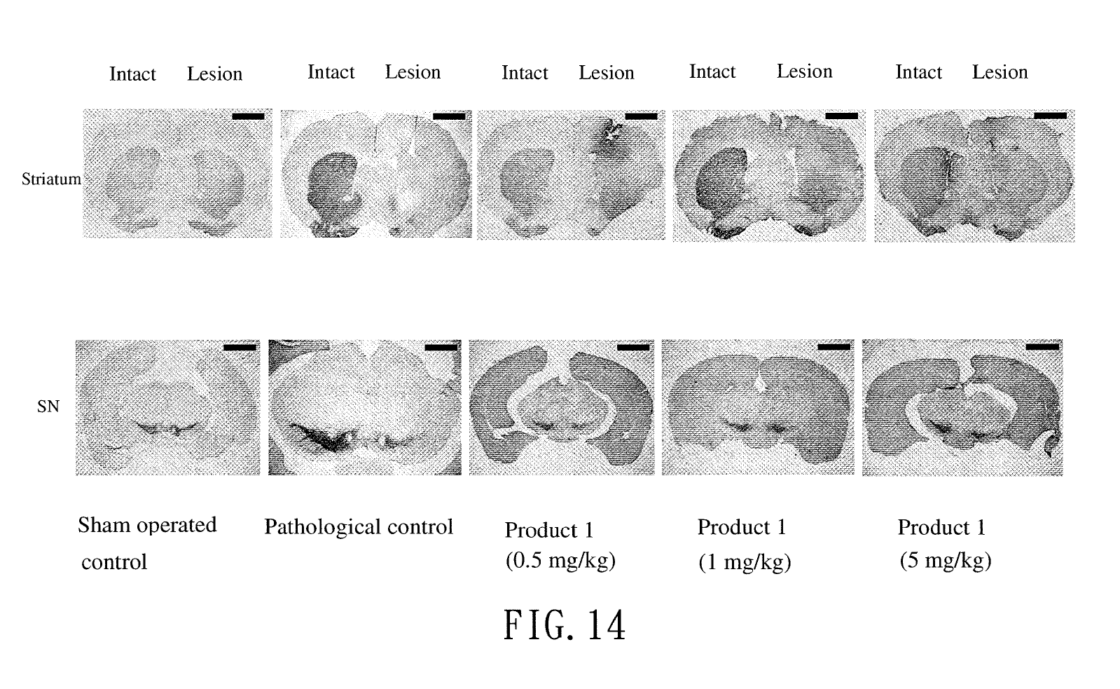

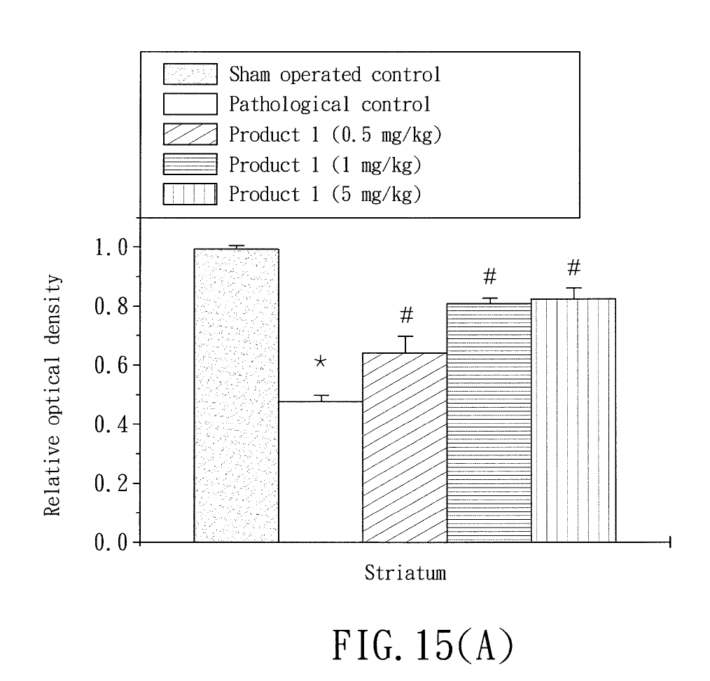

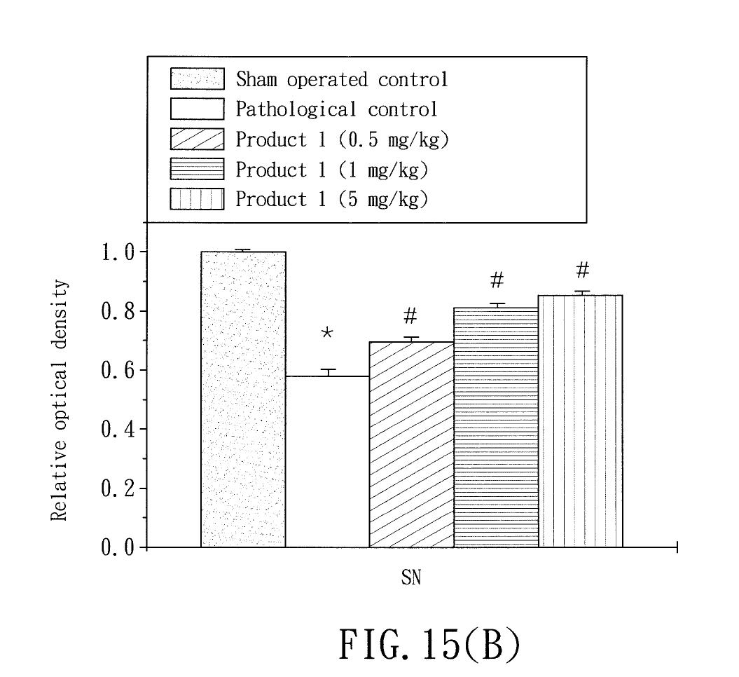

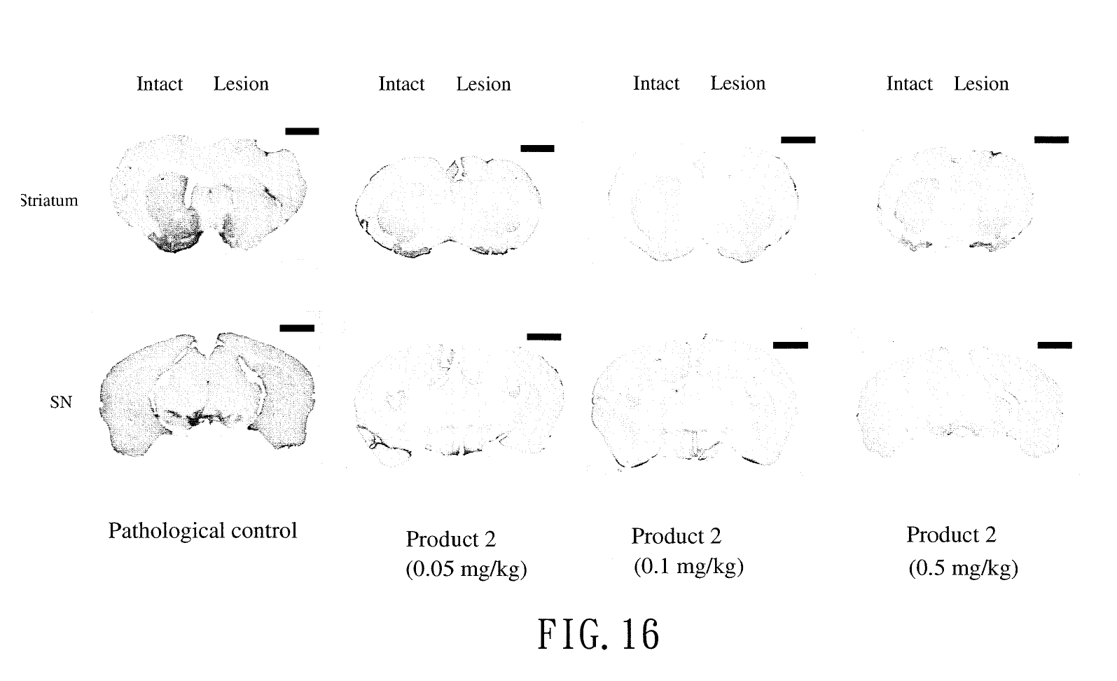

As shown in FIG. 14, and FIGS. 15(A) and 15(B), significant loss of TH-ir fibers in both of the striatum and SN was found on Day 21 after the 6-OHDA injection, but product 1 significantly alleviated the loss of TH-ir fibers in both of the striatum and SN. As shown in FIG. 16, significant loss of TH-ir fibers in both of the striatum and SN was found on Day 21 after the 6-OHDA injection, but product 2 significantly alleviated the loss of TH-ir fibers in both of the striatum and SN.

In view of the results of FIGS. 13(A), 13(B), 14, 15(A), 15(B), and 16, it is found that there is a remarkable correlation between TH-ir fiber loss in the striatum and SN, and the rotational behavior.

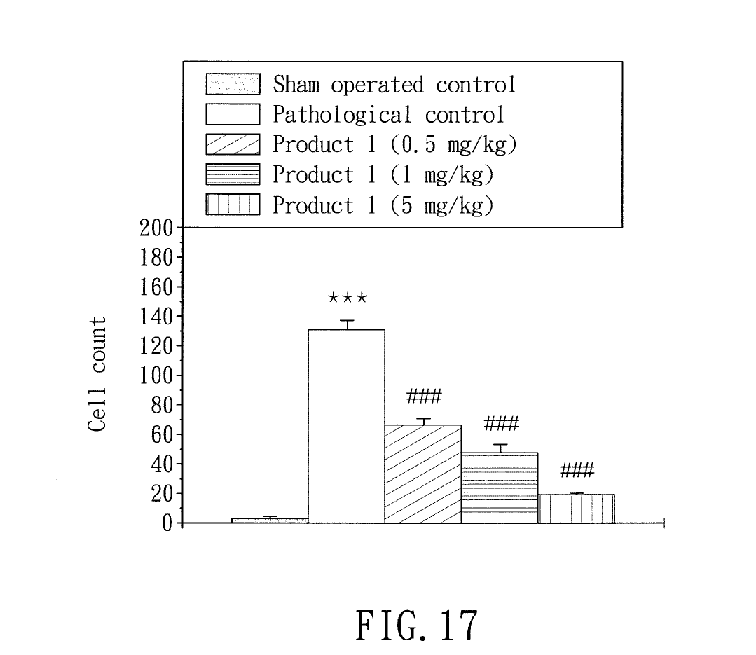

As shown in FIG. 17, iNOS expression in the striatum was significantly high on Day 21 after the 6-OHDA injection, but product 1 significantly inhibited iNOS expression in the striatum. There is a dose-dependent relationship between the dose of product 1 and the number of iNOS-positive cells in the striatum.

The aforesaid experimental results reveal that the subchronic post-treatments with the biphenolic compounds of formula (I) are able to alleviate the rotational behavior and reduction in TH expression induced by 6-OHDA, and are capable of inhibiting iNOS expression induced by 6-OHDA.

C. Effect of Treatment with Biphenolic Compound of Formula (I) on Incoordination Induced by 6-OHDA
1. Pretreatment with Biphenolic Compound of Formula (I)

Product 1 was administered intraperitoneally to NMRI mice at a dose of 5 mg/kg. 30 minutes after the administration of product 1, the NMRI mice were subjected to 6-OHDA injection according to the method as described in section 2 of General Procedures.

NMRI mice into which the vehicle was injected, and which were not treated with product 1 before the injection of the vehicle, served as a sham operated control. NMRI mice that were subjected to 6-OHDA injection and that were not treated with product 1 before the 6-OHDA injection served as a pathological control.

The NMRI mice were subjected to the beam walking test as described in the following subsection 3 on Day 1 and Day 7 after the injection of 6-OHDA or the vehicle. The obtained data were subjected to the statistical analysis as described in section 5 of General Procedures.

2. Post-Treatments with Biphenolic Compound of Formula (I)

NMRI mice were subjected to 6-OHDA injection according to the method as described in section 2 of General Procedures. On Day 7 after the 6-OHDA injection, product 1 was administered intraperitoneally to the NMRI mice at a dose of 5 mg/kg respectively for 7 and 14 consecutive days.

NMRI mice into which a vehicle (saline containing 0.02% ascorbic acid) was injected (generally according to the method as described in section 2 of General Procedures, except that 6-OHDA was not injected), and which were not treated with product 1 after the injection of the vehicle, served as a sham operated control. NMRI mice that were subjected to 6-OHDA injection and that were not treated with product 1 after the 6-OHDA injection served as a pathological control.

The NMRI mice were subjected to the beam walking test as described in the following subsection 3 on Day 14 or Day 21 after the injection of 6-OHDA or the vehicle. The obtained data were subjected to the statistical analysis as described in section 5 of General Procedures.

### 3. Beam Walking Test

A beam walking test was conducted under proper conditions, e.g., silence and illumination, so as to evaluate coordination of the respective NMRI mouse. The respective NMRI mouse was placed at a starting point of a beam having a height of 50 cm from a supporting surface, equipped with an escape plateform at each end. The beam had a length of 60 cm and a diameter of 1.2 cm. Before the beam walking test, the respective mouse was trained to walk through the beam for 3 days so that the escape latency thereof was less than 20 seconds. The total travel distance was measured.

### Results:

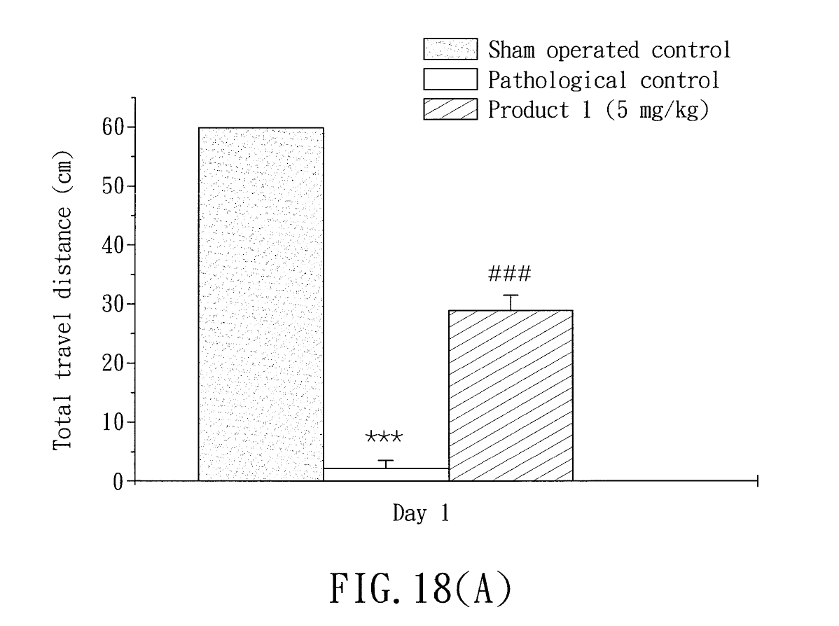

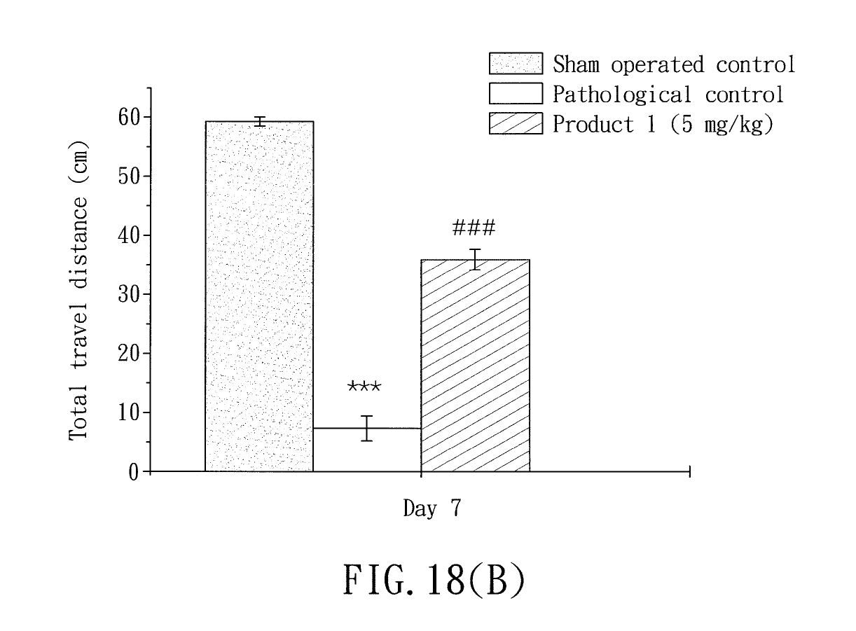

As shown in FIGS. 18(A) and 18(B), the pretreatment with product 1 significantly alleviated the motor incoordination, and the effect of the pretreatment with product 1 on the motor incoordination can last for at least 7 days.

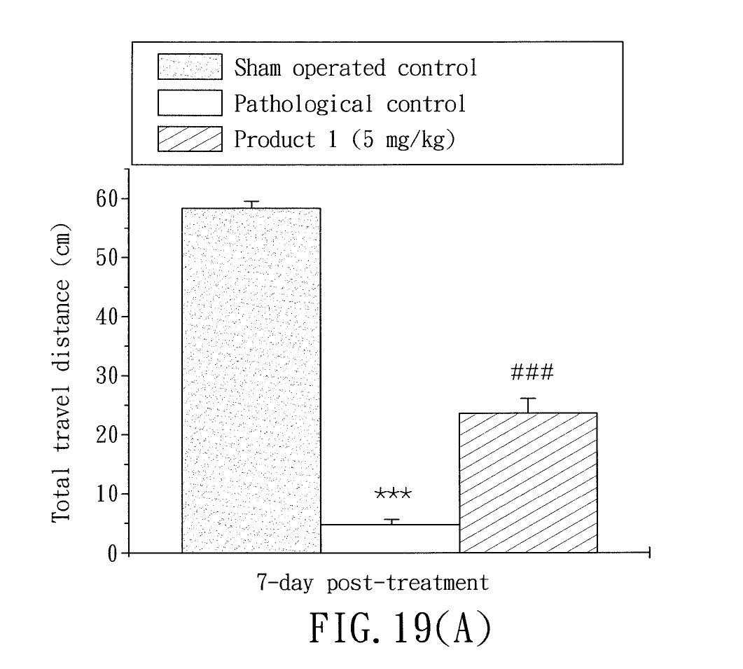

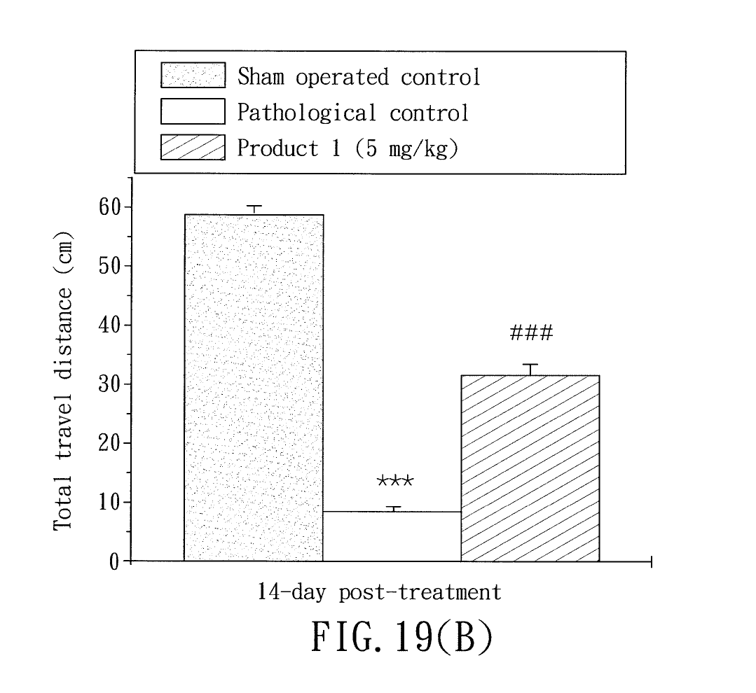

As shown in FIG. 19, the post-treatments with product 1 for 7 days and 14 days significantly alleviated the motor incoordination, and the longer post-treatment with product 1 (i.e., 14 days) seems to be capable of more effectively alleviating the motor incoordination compared to the shorter post-treatment with product 1 (i.e., 7 days).

In view of the results of Pharmacological Examples 1 and 2, the biphenolic compounds of formula (I) are effective in treating Parkinson's disease. The pharmaceutical composition for treating Parkinson's disease according to the present invention is expected to be useful.

All patents and literature references cited in the present specification as well as the references described therein, are hereby incorporated by reference in their entirety. In case of conflict, the present description, including definitions, will prevail.

While the invention has been described with reference to the above specific embodiments, it is apparent that numerous modifications and variations can be made without departing from the scope and spirit of this invention. It is therefore intended that this invention be limited only as indicated by the appended claims.

We claim:

1. A method of producing a biphenolic compound of formula (I): 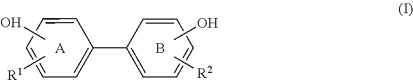
wherein R¹ in ring A and R² in ring B independently represent a C₁-C₁₂ alkyl group, a C₂-C₁₂ alkenyl group, or a C₂-C₁₂ alkynyl group;
the method comprising:
subjecting an anisole compound of formula (II) and an arylboronic compound of formula (III) to Suzuki reaction so that a biphenyl compound of formula (I′) is formed: 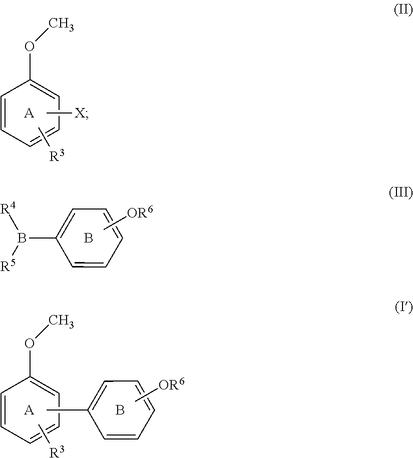
wherein, in formula (II), X represents halogen, and, in formulas (II) and (I′), R³ is H, an optionally substituted C₁-C₁₂ alkyl group, a C₂ or C₄-C₁₂ terminal alkenyl group, or a —(CH₂)ₙ—CH(OH)CH₂OH group, n being an integer from 1-10;
wherein, in formula (III), R⁴ and R⁵ represent OH, or R⁴ and R⁵ together with the boron atom to which R⁴ and R⁵ are attached form boronic ester; and, in formulas (III) and (I′), R⁶ represents tetrahydropyranyl,
removing R⁶ from the biphenyl compound of formula (I′), followed by attaching a R⁷ group to the ring B, R⁷ having the same definition as R²; and
converting the methoxy group in the ring A to a hydroxy group.

2. The method of claim 1, wherein: when R³ is H or the —(CH₂)ₙ—CH(OH)CH₂OH, the method further comprises converting R³ to R¹.

3. The method of claim 1, wherein R¹ and R² independently represent propyl, propenyl, propynyl, 2-methylpropyl, 2,2-dimethylpropyl, butyl, pentyl, hexyl, 3-butenyl, or 4-pentenyl.

4. The method of claim 1, wherein R³ is selected from the group consisting of propyl, 2,3-dihydroxypropyl, 2-methylpropyl, 2,2-dimethylpropyl, butyl, pentyl, hexyl, 3-butenyl, and 4-pentenyl.

5. The method of claim 1, wherein X is Br.

6. The method of claim 5, wherein the anisole compound is selected from 3-(3-bromo-4-methoxy-phenyl)-propane-1,2-diol, 2-bromo-4-propyl anisole, 3-bromo anisole, 4-bromo-5-propyl anisole, and 3-(2-bromo-5-methoxy-phenyl)-propane-1,2-diol.

7. The method of claim 1, wherein R⁴ and R⁵ together with the boron atom to which R⁴ and R⁵ are attached form boronic acid pinacol ester.

8. The method of claim 7, wherein the arylboronic compound is 4-(tetrahydro-2H-pyran-2-yloxy)-phenylboronic acid pinacol ester.

9. A biphenyl compound of formula (I′): 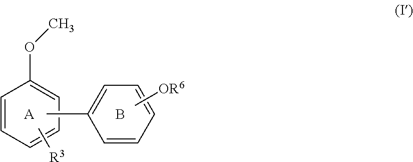
wherein R³ is H, an optionally substituted C₁-C₁₂ alkyl group, a C₂ or C₄-C₁₂ terminal alkenyl group, or a —(CH₂)ₙ—CH(OH)CH₂OH group, n being an integer from 1-10, and R⁶ is tetrahydropyranyl.

10. The biphenyl compound of claim 9, wherein R³ is selected from the group consisting of propyl, 2,3-dihydroxypropyl, 2-methylpropyl, 2,2-dimethylpropyl, butyl, pentyl, hexyl, 3-butenyl, and 4-pentenyl.

11. The biphenyl compound of claim 9, which is selected from 3-[6-methoxy-4′-(tetrahydro-pyran-2-yloxy)-biphenyl-3-yl]-propane-1,2-diol, 2-(2′-methoxy-5′-propyl-biphenyl-4-yloxy)-tetrahydro-pyran, 2-(3′-methoxy-biphenyl-4-yloxy)-tetrahydro-pyran, 2-(4′-methoxy-2′-propyl-biphenyl-4-yloxy)-tetrahydro-pyran, and 2-[4-methoxy-4′-(tetrahydro-pyran-2-yloxy)-biphenyl-3-yl]-propane-1,2-diol.

12. A method of producing a biphenyl compound of formula (I′): 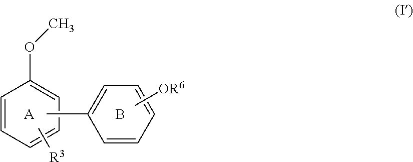
the method comprising:
subjecting an anisole compound of formula (II) and an arylboronic compound of formula (III) to Suzuki reaction: 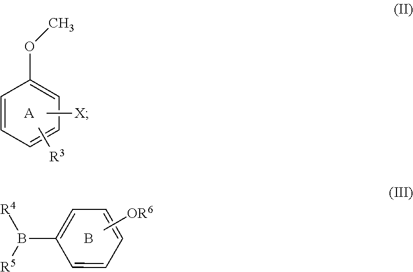
wherein, in formula (II), X represents halogen, and, in formulas (II) and (I′), R³ is H, an optionally substituted C₁-C₁₂ alkyl group, a C₂ or C₄-C₁₂ terminal alkenyl group, or a —(CH₂)ₙ—CH(OH)CH₂OH group, n being an integer from 1-10;
wherein, in formula (III), R⁴ and R⁵ represent OH, or R⁴ and R⁵ together with the boron atom to which R⁴ and R⁵ are attached form boronic ester; and, in formulas (III) and (I′), R⁶ represents tetrahydropyranyl.

13. The method of claim 12, wherein R³ is selected from the group consisting of propyl, 2,3-dihydroxypropyl, 2-methylpropyl, 2,2-dimethylpropyl, butyl, pentyl, hexyl, 3-butenyl, and 4-pentenyl.

14. The method of claim 12, wherein X is Br.

15. The method of claim 14, wherein the anisole compound is selected from 3-(3-bromo-4-methoxy-phenyl)-propane-1,2-diol, 2-bromo-4-propyl anisole, 3-bromo anisole, 4-bromo-5-propyl anisole, and 3-(2-bromo-5-methoxy-phenyl)-propane-1,2-diol.

16. The method of claim 12, wherein R⁴ and R⁵ together with the boron atom to which R⁴ and R⁵ are attached form boronic acid pinacol ester.

17. The method of claim 16, wherein the arylboronic compound is 4-(tetrahydro-2H-pyran-2-yloxy)-phenylboronic acid pinacol ester.

18. A pharmaceutical composition for treating Parkinson's disease, comprising the biphenolic compound of formula (I) as defined in claim 1.

19. The pharmaceutical composition of claim 18, wherein the biphenolic compound of formula (I) is selected from the group consisting of 5,3′-diallyl-biphenyl-2,4′-diol, 3′-allyl-5-propyl-biphenyl-2,4′-diol, 5,3′-dipropyl-biphenyl-2,4′-diol, 5-allyl-3′-propyl-biphenyl-2,4′-diol, 3′-allyl-5-prop-2-ynyl-biphenyl-2,4′-diol, 2,3′-diallyl-biphenyl-3,4′-diol, and 3,3′-diallyl-biphenyl-4,4′-diol.

20. The pharmaceutical composition of claim 19, wherein the biphenolic compound of formula (I) is 5,3′-diallyl-biphenyl-2,4′-diol.

21. The pharmaceutical composition of claim 19, wherein the biphenolic compound of formula (I) is 3′-allyl-5-propyl-biphenyl-2,4′-diol
